(19) United States
(12) Patent Application Publication
(10) Pub. No.: US 2019/0247558 A1
(43) Pub. Date: Aug. 15, 2019

(54) BLEEDING CONDUIT ASSEMBLY

(71) Applicant: SUN MEDICAL TECHNOLOGY RESEARCH CORPORATION, Suwa-shi, Nagano (JP)

(72) Inventors: Takayuki MIYAKOSHI, Nagano (JP); Kenji YAMAZAKI, Hokkaido (JP); Shinji KOBAYASHI, Nagano (JP); Hideki KANEBAKO, Nagano (JP)

(21) Appl. No.: 16/329,217

(22) PCT Filed: Aug. 29, 2017
(86) PCT No.: PCT/JP2017/031023

§ 371 Date: Feb. 28, 2019

(30) Foreign Application Priority Data
Aug. 29, 2016 (JP) PCT/JP2016/075248

(51) Int. Cl. A61M 1/10; A61L 27/18; A61L 27/56; A61M 1/12

(52) U.S. Cl. CPC A61M 1/1008; A61L 27/18; A61M 2205/04; A61M 1/122; A61L 27/56

(57) ABSTRACT

Provided is a bleeding conduit assembly attached to a heart and used for introducing blood in the heart into a ventricular assist pump from the heart. The bleeding conduit assembly includes: a bleeding conduit made of a porous material; and a cuff mounted on one end portion of the bleeding conduit, wherein a first gas non-permeable material layer is formed on an outer peripheral surface of the bleeding conduit. The bleeding conduit assembly according to the present invention can overcome at least any one of three drawbacks which occur attributed to the presence of an inflow cannula in a conventional bleeding conduit assembly.

### RELATED APPLICATIONS

The present application is a National Phase of International Application Number PCT/JP2017/031023, filed Aug. 29, 2017, which claims priority to International Application Number PCT/JP2016/075248, filed Aug. 29, 2016.

### TECHNICAL FIELD

The present invention relates to a “bleeding conduit assembly”, a “ventricular assist pump system (artificial ventricular assist pump system)” and a “method of attaching a bleeding conduit assembly to a heart”.

### BACKGROUND ART

There has been known a bleeding conduit assembly which is attached to a heart and used for introducing blood in the heart into a ventricular assist pump (artificial ventricular assist pump) from the heart (see patent literature 1, for example).

FIG. 16 and FIG. 17 are views for describing a bleeding conduit assembly 900 described in patent literature 1. FIG. 16 is a view showing a use example of a bleeding conduit assembly 900 described in patent literature 1, and FIG. 17 is a view showing the structure of the bleeding conduit assembly 900 described in patent literature 1. In FIG. 16, symbol 12 indicates an outflow graft, symbol 14 indicates a pump cable, symbol 20 indicates a body of a patient, and symbol 28 indicates a thoracic cavity. In FIG. 17, symbol 942 indicates a fastening clamp.

As shown in FIG. 16, the bleeding conduit assembly 900 described in patent literature 1 is a bleeding conduit assembly which is attached to a heart and used for introducing blood in the heart into a ventricular assist pump from the heart. The bleeding conduit assembly 900 described in patent literature 1 is also a bleeding conduit assembly which connects a ventricular assist pump 10 and an apex cordis 24 of a left ventricle 22 to each other. As shown in FIG. 17, the bleeding conduit assembly 900 described in patent literature 1 includes: an artificial blood vessel (bleeding conduit) 910; an inflow cannula 920 disposed on one end portion of the artificial blood vessel 910; a reinforcing ring (also referred to as a reinforcing helix) 930 disposed on an outer peripheral portion of the artificial blood vessel 910; a tubular connecting member (not shown in the drawing) disposed on the other end portion of the artificial blood vessel 910; and a connecting ring 940.

A cuff 922 is disposed on an outer peripheral portion of the inflow cannula 920. In the bleeding conduit assembly 900 described in patent literature 1, a blood contact surface of the inflow cannula 920 is formed of a porous structural body made of metal wires. In this specification, the inflow cannula is also referred to as a cannula chip.

In the bleeding conduit assembly 900 described in patent literature 1, the blood contact surface of the inflow cannula 920 is formed of the porous structural body made of metal wires. Accordingly, in steps of using the bleeding conduit assembly 900, thrombus is anchored by the porous structural body and, at the same time, endothelial cells are stably fixed to such a portion. As a result, it is possible to provide a bleeding conduit assembly which can reduce a problem relating to the occurrence of thrombus compared to conventional bleeding conduit assemblies (for example, see patent literatures 2 and 3).

### CITATION LIST

### Patent Literature

PTL 1: JP 2010-104428 A

PTL 2: JP 2005-124859 A

PTL 3: JP 2005-080991 A

### SUMMARY OF INVENTION

### Technical Problem

FIG. 18 and FIG. 19 are views for describing a drawback of the conventional bleeding conduit assembly.

As described above, the bleeding conduit assembly 900 described in patent literature 1 is an excellent bleeding conduit assembly which can reduce a problem relating to the occurrence of thrombus compared to the conventional bleeding conduit assemblies. However, from studies which inventors of the present invention have made, it is found that even such an excellent bleeding conduit assembly 900 described in patent literature 1 cannot sufficiently solve the following three drawbacks (1) to (3) which the conventional bleeding conduit assemblies have.

(1) The drawback (1) that, in the case where a bleeding conduit assembly is attached to a heart of a patient having an extremely weak blood flow 30 in a heart, as shown in FIG. 18, there is a concern that thrombus 32 occurs at a root portion of the inflow cannula 920

(2) The drawback (2) that, in the case where the bleeding conduit assembly 900 is brought into a state where the bleeding conduit assembly 900 is attached to a heart obliquely with respect to the heart due to an operation or contraction of the heart after an operation, as shown in FIG. 19, there is a concern that thrombus 32 occurs at a portion where the inflow cannula 920 and a myocardium 26 (or an inner wall surface 34 of a heart) are disposed close to each other or are brought into contact with each other

(3) The drawback (3) that, in the case where a pressure in a heart chamber becomes a negative pressure (less than 1 atmospheric pressure), the blood flow 30 becomes weak so that there is a concern that thrombus 32 occurs at a root portion of the inflow cannula 920 or a concern that thrombus 32 occurs at a portion where the inflow cannula 920 and the myocardium 26 are disposed close to each other or are brought into contact with each other.

The present invention has been made in view of the above-mentioned circumstances, and it is an object of the present invention to provide a bleeding conduit assembly which can overcome at least one of the above-mentioned three drawbacks.

It is another object of the present invention to provide a ventricular assist pump system which includes such an excellent bleeding conduit assembly.

It is still another object of the present invention to provide a method of attaching such an excellent bleeding conduit assembly to a heart.

### Solution to Problem

A bleeding conduit assembly according to the present invention is a bleeding conduit assembly attached to a heart and used for introducing blood in the heart into a ventricular assist pump from the heart, the bleeding conduit assembly including: a bleeding conduit made of a porous material; and a cuff mounted on one end portion of the bleeding conduit, wherein a first gas non-permeable material layer is formed on an outer peripheral surface of the bleeding conduit.

That is, the bleeding conduit assembly according to the present invention includes the bleeding conduit and the cuff mounted on one end portion of the bleeding conduit, while the bleeding conduit assembly according to the present invention does not include an inflow cannula (cannula chip) which a conventional bleeding conduit assembly includes. Accordingly, the inflow cannula (cannula chip) does not exist in the bleeding conduit assembly of the present invention.

In this manner, the bleeding conduit assembly according to the present invention does not include the inflow cannula. Accordingly, it is possible to prevent the occurrence of

a state where thrombus occurs at a root portion of the inflow cannula in the case where the bleeding conduit assembly is attached to a heart of a patient having an extremely weak blood flow in the heart,

a state where thrombus occurs at a portion where the inflow cannula and a myocardium are disposed close to each other or are brought into contact with each other in the case where the bleeding conduit assembly is brought into a state where the bleeding conduit assembly is attached to a heart obliquely with respect to the heart due to an operation or contraction of the heart after an operation, and

a state where even in the case where a pressure in a heart chamber becomes a negative pressure (less than 1 atmospheric pressure), the blood flow 30 becomes weak so that thrombus 32 occurs at a root portion of the inflow cannula 920 or thrombus 32 occurs at a portion where the inflow cannula 920 and the myocardium 26 are disposed close to each other or are brought into contact with each other. That is, it is possible to overcome the above-mentioned three drawbacks.

As described above, the inflow cannula does not exist in the bleeding conduit assembly according to the present invention. However, it is confirmed by a test (see an test example described later) carried out by inventors of the present invention that, in the same manner as the bleeding conduit assembly 900 described in patent literature 1, the bleeding conduit assembly can be properly attached to a heart, and it is possible to prevent the occurrence of a state where an opening formed in the heart by an operation is gradually narrowed after an operation attributed to attaching the bleeding conduit assembly according to the present invention to the heart.

According to the bleeding conduit assembly of the present invention, the bleeding conduit is made of a porous material. Accordingly, in a step of using the bleeding conduit (bleeding conduit assembly), thrombus is anchored on an inner peripheral surface of the bleeding conduit made of a porous material so that a pseudo inner membrane (quasi inner membrane) is formed, and endothelial cells are stably fixed to a part near a heart (a part within 2 to 3 cm from the heart). As a result, it is possible to provide the bleeding conduit assembly which can reduce a problem of the occurrence of thrombus.

According to the bleeding conduit assembly of the present invention, the first gas non-permeable material layer is formed on the outer peripheral surface of the bleeding conduit. Accordingly, in spite of the fact that the bleeding conduit made of a porous material is used, it is possible to prevent entrainment of air into the bleeding conduit even when a pressure in the bleeding conduit becomes a negative pressure (less than 1 atmospheric pressure).

In this specification, the bleeding conduit (and the bleeding conduit assembly) is a medical part attached to a heart and used for introducing blood in the heart into a ventricular assist pump from the heart. The cuff is a member for attaching the bleeding conduit assembly to the heart. The inflow cannula is a member used in a state where the inflow cannula is attached to a distal end portion of an artificial blood vessel, and is a member having a portion which protrudes into the heart.

In the bleeding conduit assembly according to the present invention, it is preferable that the bleeding conduit be formed of an artificial blood vessel made of stretchable porous polytetrafluoroethylene.

According to the bleeding conduit assembly of this mode, the bleeding conduit is formed of an artificial blood vessel made of stretchable porous polytetrafluoroethylene (hereinafter referred to as ePTFE) having excellent flexibility and hence, the bleeding conduit assembly can be easily connected to the ventricular assist pump. According to the bleeding conduit assembly of this mode, the bleeding conduit is formed of an artificial blood vessel made of stretchable porous polytetrafluoroethylene having excellent flexibility and hence, a burden imposed on a human body can be reduced. According to the bleeding conduit assembly of this mode, the bleeding conduit is formed of an artificial blood vessel made of stretchable porous polytetrafluoroethylene and hence, it is possible to provide the bleeding conduit assembly having excellent blood compatibility and excellent antithrombogenicity.

In the bleeding conduit assembly according to the present invention, it is also preferable that the bleeding conduit be formed of an artificial blood vessel made of a polyester woven fabric.

According to the bleeding conduit assembly of this mode, the bleeding conduit is formed of the artificial blood vessel made of polyester woven fabric having excellent flexibility and hence, the bleeding conduit assembly can be easily connected to the ventricular assist pump. According to the bleeding conduit assembly of this mode, the bleeding conduit is formed of the artificial blood vessel made of polyester woven fabric having excellent flexibility and hence, a burden imposed on a human body can be reduced. According to the bleeding conduit assembly of this mode, the bleeding conduit is formed of the artificial blood vessel made of polyester woven fabric and hence, it is possible to provide the bleeding conduit assembly having excellent blood compatibility and excellent antithrombogenicity.

In the bleeding conduit assembly according to the present invention, it is preferable that the cuff be made of a polytetrafluoroethylene non-woven fabric (hereinafter referred to as a PTFE non-woven fabric).

According to the bleeding conduit assembly of this mode, the cuff is made of a PTFE non-woven fabric and hence, it is possible to provide the bleeding conduit assembly having excellent biocompatibility with a heart tissue. According to the bleeding conduit assembly of this mode, the cuff is made of a PTFE non-woven fabric and hence, the cuff has high flexibility whereby the degree of freedom of an operation performed at the time of attaching the bleeding conduit assembly to a heart is increased. It is possible to realize the bleeding conduit assembly having a shape which conforms to a shape and a structure of a heart of a patient. Further, in performing an operation, thread bridging is performed to pledgets, the heart, and the cuff respectively using suture threads and, thereafter, the suture threads are tied. Accordingly, it is possible to realize the bleeding conduit assembly having a shape such that the cuff covers a cross section (for example, a punching cut surface) of an opening formed in a heart by an operation (see FIG. 4D to FIG. 4F described later). In the bleeding conduit assembly of this mode, the cuff is not limited to a cuff made of a PTFE non-woven fabric, and may be a cuff made of a polyester non-woven fabric or the like.

In an operation performed at the time of attaching the bleeding conduit assembly of this mode to a heart, a monofilament suture thread made of PTFE, a multifilament suture thread made of PTFE, a multifilament suture thread made of polyester or the like can be used. Among these suture threads, it is preferable to use the multifilament suture thread made of polyester.

In the bleeding conduit assembly of the present invention, it is preferable that the first gas non-permeable material layer be formed of a polyurethane layer or a polycarbonate urethane layer.

According to the bleeding conduit assembly of this mode, the first gas non-permeable material layer is formed of a polyurethane layer or a polycarbonate urethane layer and hence, the bleeding conduit assembly can acquire an excellent gas non-permeable performance. As a result, the bleeding conduit assembly can acquire an excellent air entrainment prevention performance. The first gas non-permeable material layer is formed of a polyurethane layer or a polycarbonate urethane layer and hence, the first gas non-permeable material layer does not adversely affect a living body.

In this case, it is preferable that a thickness of the polyurethane layer or the polycarbonate urethane layer fall within a range of 0.05 mm to 0.5 mm inclusive. When the thickness is smaller than 0.05 mm, gas non-permeable performance is lowered so that there may be a case where the bleeding conduit assembly cannot acquire a sufficient air entrainment prevention effect. On the other hand, when the thickness is larger than 0.5 mm, flexibility of the bleeding conduit is lowered and hence, operability at the time of connecting the bleeding conduit assembly to the ventricular assist pump is lowered or a burden imposed on a human body is increased. From these viewpoints, it is preferable that the thickness of the polyurethane layer or the polycarbonate urethane layer fall within a range of 0.1 mm to 0.4 mm inclusive. It is more preferable that the thickness fall within a range of 0.15 mm to 0.25 mm inclusive.

In the bleeding conduit assembly of the present invention, it is preferable that a second gas non-permeable material layer be formed in at least an inner-peripheral-side predetermined region on a surface of the cuff on a bleeding conduit side.

According to the bleeding conduit assembly of this mode, the second gas non-permeable material layer is formed in at least the inner-peripheral-side predetermined region on the surface of the cuff on the bleeding conduit side (see FIG. 4E described later). Accordingly, when the bleeding conduit assembly of this mode is attached to a heart, it is possible to suppress as much as possible the occurrence of a state where air is entrained from the inside of a heart through the cuff.

In the bleeding conduit assembly according to the present invention, it is preferable that the first gas non-permeable material layer and the second gas non-permeable material layer be formed continuously.

According to the bleeding conduit assembly of this mode, the first gas non-permeable material layer and the second gas non-permeable material layer are formed continuously. Accordingly, when the bleeding conduit assembly of this mode is attached to a heart, it is possible to suppress as much as possible the occurrence of a state where air is entrained from the inside of a heart through the cuff.

In the bleeding conduit assembly according to the present invention, it is preferable that the second gas non-permeable material layer be formed within a region of at least 2 mm from an outer peripheral surface of the bleeding conduit.

According to the bleeding conduit assembly of this mode, the second gas non-permeable material layer is formed within the region of at least 2 mm from the outer peripheral surface of the bleeding conduit (a region indicated by symbol A in FIG. 2C described later). Accordingly, when the bleeding conduit assembly of this mode is attached to a heart, it is possible to narrow as much as possible a path when air is entrained from the inside of a heart through the cuff.

In the bleeding conduit assembly of the present invention, it is preferable that the second gas non-permeable material layer be not formed within a region of at least 2 mm from an outer periphery of the cuff.

According to the bleeding conduit assembly of this mode, the second gas non-permeable material layer is not formed within the region of at least 2 mm from the outer periphery of the cuff (a region indicated by symbol B in FIG. 2C described later). Accordingly, when the bleeding conduit assembly of this mode is attached to a heart, a tissue of a living body infiltrates into the cuff also on a surface of the cuff on a bleeding conduit side and hence, the cuff and the living body are favorably adhered to each other.

In the bleeding conduit assembly according to the present invention, it is preferable that the second gas non-permeable material layer be formed of a polyurethane layer or a polycarbonate urethane layer.

According to the bleeding conduit assembly of this mode, the second gas non-permeable material layer is formed of a polyurethane layer or a polycarbonate urethane layer and hence, the bleeding conduit assembly can acquire an excellent gas non-permeable performance. As a result, the bleeding conduit assembly can acquire an excellent air entrainment prevention performance. The second gas non-permeable material layer is formed of a polyurethane layer or a polycarbonate urethane layer and hence, the second gas non-permeable material layer does not adversely affect a living body.

Also in the case of the second gas non-permeable material layer, it is preferable that a thickness of the polyurethane layer or the polycarbonate urethane layer fall within a range of 0.05 mm to 0.5 mm inclusive. When the thickness is smaller than 0.05 mm, gas non-permeable performance is lowered so that there may be a case where the bleeding conduit assembly cannot acquire a sufficient air entrainment prevention effect. When the thickness is larger than 0.5 mm, flexibility of the cuff is lowered and hence, operability at the time of attaching the bleeding conduit assembly to a heart is lowered or there may be a case where it is difficult to realize a state where the cuff covers a cross section (for example, a punching cut surface) of an opening formed in a heart by an operation. From these viewpoints, it is preferable that the thickness of the polyurethane layer or the polycarbonate urethane layer fall within a range of 0.1 mm to 0.4 mm inclusive. It is more preferable that the thickness fall within a range of 0.15 mm to 0.25 mm inclusive.

In the bleeding conduit assembly according to the present invention, it is preferable that the cuff be sutured to the bleeding conduit.

According to the bleeding conduit assembly of this mode, the cuff is sutured to the bleeding conduit and hence, flexible connection between the cuff and the bleeding conduit can be realized. Accordingly, the bleeding conduit assembly having a shape which conforms to a shape and a structure of a heart of a patient can be realized.

In the bleeding conduit assembly according to this mode, in suturing the cuff to the bleeding conduit, a monofilament suture thread made of PTFE, a multifilament suture thread made of PTFE, a multifilament suture thread made of polyester or the like can be used. Among these suture threads, it is preferable to use the multifilament suture thread made of polyester. In the bleeding conduit assembly of this mode, after the cuff is sutured to the bleeding conduit, the cuff may be further firmly mounted on the bleeding conduit using an adhesive agent.

In the bleeding conduit assembly according to the present invention, it is preferable that a width W of the cuff in a diametric direction fall within a range of 5 mm to 16 mm inclusive.

In the bleeding conduit assembly of this mode, the reason that the above-mentioned width W is set to a value which falls within a range of 5 mm to 16 mm inclusive is as follows. That is, when the above-mentioned width W is smaller than 5 mm, a width of the cuff when the bleeding conduit assembly is attached to a heart is too narrow so that there may be a case where a cross section (punched surface) of an opening formed in a heart cannot be sufficiently covered by the cuff (see FIG. 4D and FIG. 4E described later, the punched surface being sufficiently covered by the cuff in FIG. 4D and FIG. 4E), or an operation of bridging a suture thread between a heart and a cuff in a spiral shape cannot be performed (see FIG. 4E described later, a suture thread is correctly bridged in a spiral shape at the portion shown in FIG. 4E). On the other hand, when the above-mentioned width W is larger than 16 mm, a width of a cuff when the bleeding conduit assembly is attached to a heart is too large so that undesired burden is imposed on a body of a patient. From this viewpoint, although it depends on a size of a heart of a patient, it is preferable that the above-mentioned width W be 7 mm or more, and it is more preferable that the width W be 8 mm or more. Further, it is preferable that the above-mentioned width W be 15 mm or less, and it is more preferable that the width W be 14 mm or less.

In the bleeding conduit assembly of this mode, the width W of the cuff along a diametric direction is a size which is obtained by dividing a size obtained by subtracting a diameter D2 of an opening of the cuff from a diameter D1 of the cuff by 2 (see FIG. 2A described later).

In the bleeding conduit assembly of this mode, it is preferable that the bleeding conduit assembly be a bleeding conduit assembly which is attached to an apex cordis of a left ventricle of the heart or an area in the vicinity of the apex cordis, and is used for introducing blood in the left ventricle of the heart into the ventricular assist pump. According to the bleeding conduit assembly of this mode, the bleeding conduit assembly can be suitably used in a ventricular assist system (artificial ventricular assist system) which can efficiently assist a heart.

In the bleeding conduit assembly of the present invention, it is preferable that an annular reinforcing member be disposed on at least one end portion of the bleeding conduit.

In the bleeding conduit assembly of this mode, an inflow cannula does not exist. Accordingly, there is a concern that a situation occurs where an opening formed in a heart by an operation is gradually narrowed after the operation. However, according to the bleeding conduit assembly of this mode, the annular reinforcing member is disposed at one end portion of the bleeding conduit and hence, it is possible to prevent the occurrence of a situation where an opening formed in a heart by an operation is gradually narrowed after the operation.

The annular reinforcing member is an annular or a coil-shaped member disposed on one end portion of the artificial blood vessel for compensating a strength of the artificial blood vessel. As the annular reinforcing member, it is possible to suitably use, for example, a conventionally known helical reinforcing ring (also referred to as an auxiliary PTFE helix) which is used by being welded to an outer peripheral portion of the artificial blood vessel along a longitudinal direction. However, the annular reinforcing member is not limited to such a reinforcing ring, and a member different from the conventionally known helical reinforcing ring can be also used. For example, an annular or a helical reinforcing ring disposed on an outer peripheral portion or an inner peripheral portion at one end portion of the artificial blood vessel or an annular or helical reinforcing ring embedded in an inner peripheral portion of the cuff may be exemplified. The annular reinforcing member may have a circular cross section, an elongated circular cross section, other polygonal cross sections such as a quadrangular cross section, or cross sections having other shapes. The annular reinforcing member may be formed in a belt shape. The annular reinforcing member may be welded to an outer peripheral portion or an inner peripheral portion of the artificial blood vessel.

A material (for example MPC polymer) having blood compatibility and antithrombogenicity may be applied to an outer peripheral surface of the bleeding conduit by coating by way of the above-mentioned first gas non-permeable material layer.

In the bleeding conduit assembly according to the present invention, it is preferable that a connecting ring for connecting the bleeding conduit to the ventricular assist pump be disposed on the other end portion of the bleeding conduit.

In the bleeding conduit assembly of this mode, the connecting ring for connecting the artificial blood vessel to the ventricular assist pump is disposed on the other end portion of the artificial blood vessel and hence, it is possible to provide the bleeding conduit assembly having excellent connection operability with the ventricular assist pump.

A bleeding conduit assembly according to the present invention is a bleeding conduit assembly attached to a heart and used for introducing blood in the heart into a ventricular assist pump from the heart, the bleeding conduit assembly including: a bleeding conduit made of a porous material; an inflow cannula disposed on one end portion of the bleeding conduit; and a cuff disposed on a predetermined position of an outer periphery of the inflow cannula, wherein a gas non-permeable material layer is formed on an outer peripheral surface of the bleeding conduit.

Among advantageous effects of the bleeding conduit assembly according to the present invention, the advantageous effect based on the configuration where the bleeding conduit is made of a porous material, and the advantageous effect obtained by forming the first gas non-permeable material layer on the outer peripheral surface of the bleeding conduit can be also acquired in the case of the above-mentioned bleeding conduit assembly, that is, the bleeding conduit assembly including the inflow cannula.

A ventricular assist pump system according to the present invention includes: a ventricular assist pump having a blood introducing portion and a blood discharging portion; a bleeding conduit assembly connecting the blood introducing portion of the ventricular assist pump and a heart to each other; and an outflow graft connecting the blood discharging portion of the ventricular assist pump and an aorta to each other, wherein the bleeding conduit assembly is anyone of the bleeding conduit assemblies of the present invention described in any one of the above-mentioned (1) to (13).

The ventricular assist pump system according to the present invention includes any one of the bleeding conduit assemblies of the present invention described in the above-mentioned (1) to (13). Accordingly, it is possible to provide the ventricular assist pump system capable of solving the previously mentioned three drawbacks which occur attributed to the presence of an inflow cannula (cannula chip) in the conventional bleeding conduit assembly.

According to the ventricular assist pump system of the present invention, the bleeding conduit is made of a porous material. Accordingly, in a step of using the ventricular assist pump system, thrombus is anchored on an inner peripheral surface of the bleeding conduit made of a porous material so that at least a pseudo inner membrane (quasi inner membrane) is formed at the portion, and endothelial cells are stably fixed to a part near a heart (a part within 2 to 3 cm from the heart). As a result, it is possible to provide the ventricular assist pump system which can reduce a problem of the occurrence of thrombus.

According to the ventricular assist pump system of the present invention, the first gas non-permeable material layer is formed on the outer peripheral surface of the bleeding conduit. Accordingly, in spite of the fact that the bleeding conduit made of a porous material is used, it is possible to prevent entrainment of air into the bleeding conduit even when a pressure in the bleeding conduit becomes a negative pressure (less than 1 atmospheric pressure).

In the ventricular assist pump system according to the present invention, it is preferable that the bleeding conduit assembly be the bleeding conduit assembly described in the above-mentioned [13] . This is because the connecting ring for connecting the bleeding conduit assembly to the ventricular assist pump is disposed in the bleeding conduit assembly and hence, it is possible to provide the ventricular assist pump system where the bleeding conduit assembly and the ventricular assist pump are connected to each other more properly.

A method of attaching a bleeding conduit assembly to a heart according to the present invention includes in the following order: a first step where a bleeding conduit assembly which is formed of a bleeding conduit formed of an artificial blood vessel and a cuff mounted on one end portion of the bleeding conduit are prepared; a second step where an opening is formed at a predetermined portion of a heart; a third step where a plurality of pledgets are disposed at positions where the pledgets surround the opening outside the heart, suture threads each having a needle on both ends respectively are prepared, the respective needles of the suture threads are made to penetrate a myocardium of the heart from surfaces of the pledgets and to reach the inside of the heart thus bridging the suture threads to the heart; a fourth step where the respective needles of each suture thread are made to pass through the opening and are taken out to the outside of the heart, and the respective needles are made to pass through the cuff of the bleeding conduit assembly so as to perform bridging of the suture threads to the cuff with respect to the respective suture threads; and a fifth step where the bleeding conduit assembly is attached to the heart in a state where a surface (plane) of the bleeding conduit and an inner wall surface of the heart become coplanar with each other by tying the respective suture threads.

According to the method of attaching a bleeding conduit assembly to a heart of the present invention, the bleeding conduit assembly of the present invention can be properly attached to the heart. Accordingly, it is possible to provide the ventricular assist pump system capable of solving the previously mentioned three drawbacks which occur attributed to the presence of an inflow cannula (cannula chip) in the conventional bleeding conduit assembly.

In the method of attaching a bleeding conduit assembly to a heart according to the present invention, it is preferable that the bleeding conduit assembly be attached to the heart in a state where the bleeding conduit does not protrude to the inside of the heart from an inner wall surface of the heart in the fifth step.

According to the method of attaching a bleeding conduit assembly to a heart of this mode, it is possible to provide the ventricular assist pump system capable of solving the previously mentioned three drawbacks which occur attributed to the presence of an inflow cannula (cannula chip) in the conventional bleeding conduit assembly.

In the method of attaching a bleeding conduit assembly to a heart of the present invention, in the fifth step, it is preferable that the bleeding conduit assembly be attached to the heart in a state where the cuff covers a punching cut surface of the opening.

According to the bleeding conduit assembly of the present invention, it is possible to provide the ventricular assist pump system capable of solving the previously mentioned three drawbacks which occur attributed to the presence of an inflow cannula (cannula chip) in the conventional bleeding conduit assembly.

It is preferable that the method of attaching a bleeding conduit assembly to a heart according to the present invention further include, after the fifth step, a sixth step where bridging of the suture threads is performed in a spiral shape between the cuff and the heart using suture threads different from the above-mentioned suture threads within a region surrounding the opening thus enabling more firm attaching the bleeding conduit assembly to the heart.

According to the bleeding conduit assembly of this mode, attaching the bleeding conduit assembly to the heart can be made more firm.

### BRIEF DESCRIPTION OF DRAWINGS

FIG. 1 is a view for describing a bleeding conduit assembly 100 according to an embodiment 1.

FIG. 2A to FIG. 2C are views for describing the bleeding conduit assembly 100 according to the embodiment 1.

FIG. 3A and FIG. 3B are views for describing the bleeding conduit assembly 100 according to the embodiment 1.

FIG. 4A to FIG. 4H are views for describing a mode where the bleeding conduit assembly 100 according to the embodiment 1 is attached to an apex cordis of a heart left ventricle.

FIG. 5 is a view for describing a bleeding conduit assembly 101 according to an embodiment 2.

FIG. 6 is a view for describing a bleeding conduit assembly 102 according to an embodiment 3.

FIG. 7 is a view for describing a bleeding conduit assembly 103 according to an embodiment 4.

FIG. 8 is a view for describing a bleeding conduit assembly 104 according to an embodiment 5.

FIG. 9 is a view for describing a bleeding conduit assembly 105 according to an embodiment 6.

FIG. 10 is a view for describing a bleeding conduit assembly 106 according to an embodiment 7.

FIG. 11 is a view for describing a bleeding conduit assembly 107 according to an embodiment 8.

FIG. 12 is a view for describing a bleeding conduit assembly 108 according to an embodiment 9.

FIG. 13A to FIG. 13F are photographs showing a mode where the bleeding conduit assembly 100 is attached to a heart in a test example.

FIG. 14A to FIG. 14C are photographs of the bleeding conduit assembly 100 in the test example after a calf was brought into a sacrificial death state after a lapse of 60 days from attaching the bleeding conduit assembly 100 to a heart of the calf.

FIG. 15 a photograph showing a cross section of a liver of a calf after the calf was brought into a sacrificial death state after a lapse of 60 days from attaching the bleeding conduit assembly 100 to a heart of the calf in the test example.

FIG. 16 is a view for describing a conventional bleeding conduit assembly 900.

FIG. 17 is a view for describing the conventional bleeding conduit assembly 900.

FIG. 18 is a view for describing a drawback of the conventional bleeding conduit assembly 900.

FIG. 19 is a view for describing a drawback of the conventional bleeding conduit assembly 900.

### DESCRIPTION OF EMBODIMENTS

Hereinafter, embodiments of the present invention are described.

### Embodiment 1

### 1. Bleeding Conduit Assembly

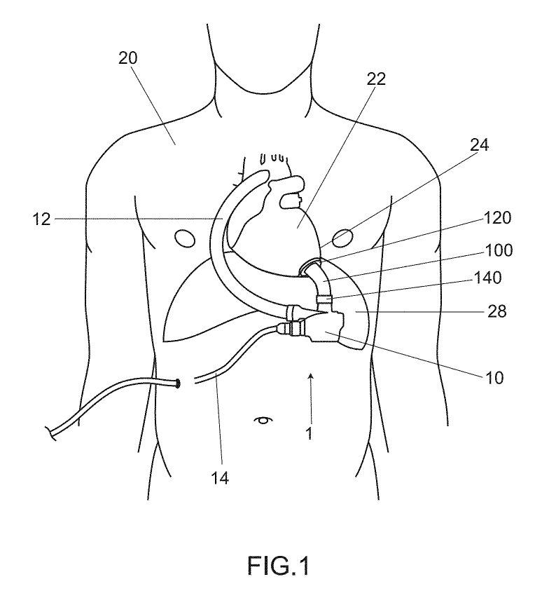

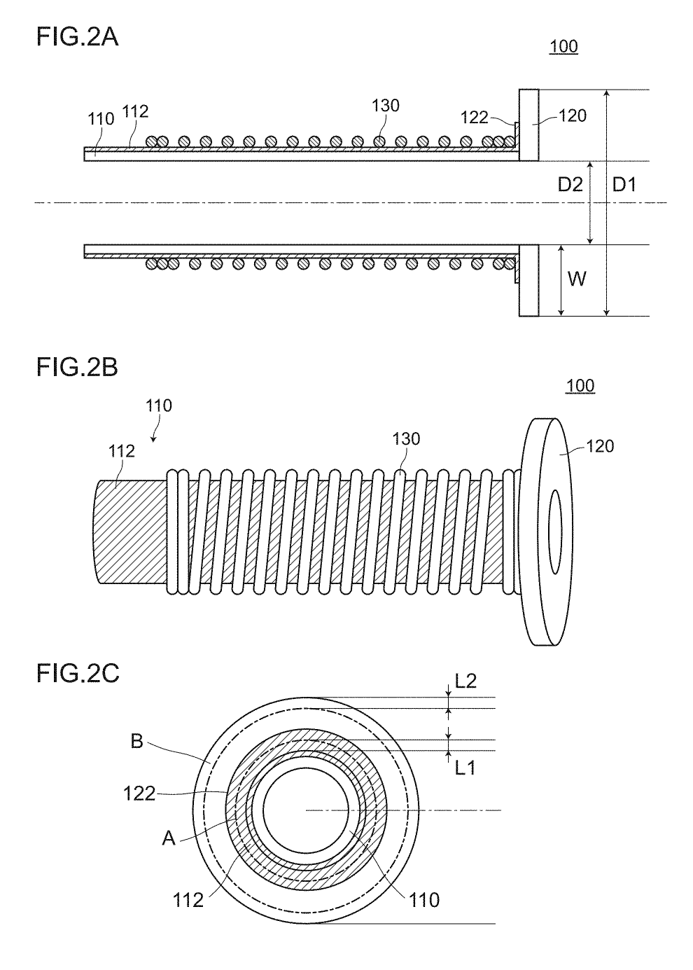

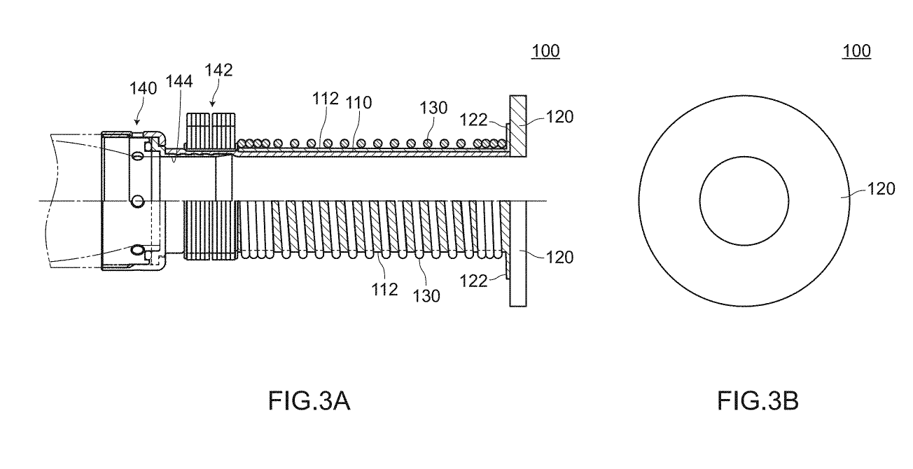

FIG. 1 to FIG. 3B are views for describing a bleeding conduit assembly 100 according to an embodiment 1. With respect to these drawings, FIG. 1 is a view showing an in-use example of the bleeding conduit assembly 100 according to the embodiment 1. FIG. 2A to FIG. 2C are views showing the structure of the bleeding conduit assembly 100 according to the embodiment 1. FIG. 2A is a cross-sectional view of the bleeding conduit assembly 100, FIG. 2B is a perspective view of the bleeding conduit assembly 100. FIG. 2C is a plan view of the bleeding conduit assembly 100 as viewed from a bleeding conduit side. FIG. 3A and FIG. 3B are views showing the structure where a connecting ring 140 is disposed on the bleeding conduit assembly 100 according to the embodiment 1. FIG. 3A is a plan view of the bleeding conduit assembly 100 with a part in cross section, and FIG. 3B is a plan view of the bleeding conduit assembly 100 as viewed from a cuff side. In FIG. 3A and FIG. 3B, symbol 142 indicates a fastening clamp, and symbol 144 indicates a tubular connecting member.

The bleeding conduit assembly 100 according to the embodiment 1 is, as shown in FIG. 1 and FIG. 2A to FIG. 2C, a bleeding conduit assembly which is attached to a heart (an apex cordis 24 of a heart left ventricle 22) and is used for introducing blood in the heart into a ventricular assist pump 10 from the heart. A ventricular assist pump system which includes the ventricular assist pump 10, the bleeding conduit assembly 100, and an outflow graft 12 which connects a blood discharging portion of the ventricular assist pump 10 and an aorta (ascending aorta) to each other is referred to as a ventricular assist pump system 1 according to the embodiment 1.

As shown in FIG. 2A to FIG. 2C, FIG. 3A and FIG. 3B, the bleeding conduit assembly 100 according to the embodiment 1 is formed of: a bleeding conduit 110 made of a porous material; and a cuff 120 mounted on one end portion of the bleeding conduit 110, wherein a first gas non-permeable material layer 112 is formed on an outer peripheral surface of the bleeding conduit 110. Accordingly, in the bleeding conduit assembly 100 according to the embodiment 1, an inflow cannula (cannula chip) does not exist unlike the case of bleeding conduit assembly 900 described in patent literature 1 or conventional bleeding conduit assemblies.

In the bleeding conduit assembly 100 according to the embodiment 1, the bleeding conduit 110 is formed of an artificial blood vessel made of stretchable porous polytetrafluoroethylene (ePTFE).

In the bleeding conduit assembly 100 according to the embodiment 1, the cuff 120 is made of a PTFE non-woven fabric. The cuff 120 is sutured to the bleeding conduit 110 using a monofilament suture thread made of PTFE, for example. In place of the monofilament suture thread made of PTFE, a multifilament suture thread made of PTFE, a multifilament suture thread made of polyester or the like can be also used.

In the bleeding conduit assembly 100 according to the embodiment 1, the first gas non-permeable material layer 112 is formed of a polycarbonate urethane layer. A thickness of the polycarbonate urethane layer falls within a range of 0.05 mm to 0.5 mm inclusive, and is 0.2 mm, for example. The polycarbonate urethane layer is formed in such a manner that a polymer solution formed by dissolving a resin pellet of polycarbonate urethane into tetrafluorofuran, for example, is applied by coating to an outer peripheral surface of the bleeding conduit by a coating method or a spray method and the polymer solution is dried. The polycarbonate urethane layer may be also formed in such a manner that a base agent which is a material for forming polycarbonate urethane and a curing agent are mixed together and, thereafter, a mixed material of the base agent and the curing agent is applied by coating to an outer peripheral surface of the bleeding conduit by a coating method or a spray method and the mixed material is cured.

In the bleeding conduit assembly 100 according to the embodiment 1, a second gas non-permeable material layer 122 is formed of a polycarbonate urethane layer. A thickness of the polycarbonate urethane layer falls within a range of 0.05 mm to 0.5 mm inclusive, and is 0.2 mm, for example. The polycarbonate urethane layer is formed in such a manner that a polymer solution formed by dissolving a resin pellet of polycarbonate urethane into tetrafluorofuran, for example is applied by coating to an outer peripheral surface of the bleeding conduit by a coating method or a spray method and the polymer solution is dried. The polycarbonate urethane layer may be also formed in such a manner that a base agent which is a material for forming polycarbonate urethane and a curing agent are mixed together and, thereafter, a mixed material of the base agent and the curing agent is applied by coating to an outer peripheral surface of the bleeding conduit by a coating method or a spray method and the mixed material is cured.

In the bleeding conduit assembly 100 according to the embodiment 1, the second gas non-permeable material layer 122 is formed in at least an inner-peripheral-side predetermined region on a surface of the cuff 120 on a bleeding conduit side.

In the bleeding conduit assembly 100 according to the embodiment 1, the first gas non-permeable material layer 112 and the second gas non-permeable material layer 122 are formed continuously.

In the bleeding conduit assembly 100 according to the embodiment 1, as shown in FIG. 2C, the second gas non-permeable material layer 122 is formed within a region A of at least 2 mm (for example, 5 mm) from an outer peripheral surface of the bleeding conduit 110. Further, the second gas non-permeable material layer 122 is not formed within a region B of at least 2 mm (for example, 3 mm) from an outer periphery of the cuff 120.

In the bleeding conduit assembly 100 according to the embodiment 1, an annular reinforcing member is disposed on (for example, welded to) one end portion of the bleeding conduit 110.

The annular reinforcing member is an annular or a coil-shaped member disposed on one end portion of the artificial blood vessel for compensating a strength of the artificial blood vessel. As the annular reinforcing member, it is possible to suitably use, for example, a conventionally known helical reinforcing ring (also referred to as an auxiliary PTFE helix) which is used by being welded to an outer peripheral portion of the artificial blood vessel along a longitudinal direction. The annular reinforcing member may be welded to an outer peripheral portion or an inner peripheral portion of the artificial blood vessel.

In the bleeding conduit assembly 100 according to the embodiment 1, a helical reinforcing ring 130 is welded to an outer peripheral portion of the bleeding conduit 110.

In the bleeding conduit assembly 100 according to the embodiment 1, as shown in FIG. 3A and FIG. 3B, a connecting ring 140 for connecting the bleeding conduit 110 to the ventricular assist pump 10 is disposed on the other end portion of the bleeding conduit 110.

In the bleeding conduit assembly 100 according to the embodiment 1, an inner diameter (an inner diameter before suturing) of the bleeding conduit 110 is 16 mm, for example. An outer diameter (an outer diameter before suturing) of the cuff 120 is 38 mm, for example, an inner diameter (an inner diameter before suturing) of the cuff 120 is 18 mm, for example. With reference to FIG. 2A, a width W (a width W before suturing) of the cuff 120 in a diametric direction falls within a range of 5 mm to 16 mm inclusive (for example, 12 mm).

As shown in FIG. 2A to FIG. 2C, the bleeding conduit assembly 100 according to the embodiment 1 has the structure where an end surface of the bleeding conduit 110 at one end portion is brought into contact with aside surface of the cuff 120 (butting structure). However, the bleeding conduit assembly 100 according to the embodiment 1 may have the structure where an outer peripheral surface of the bleeding conduit 110 at one end portion is brought into contact with an inner peripheral surface of the cuff 120 (inserting structure), or may have the structure between these two structures. In an actual operation, in a step of suturing the cuff 120 to the bleeding conduit 110, it is difficult to determine the exact connecting structure between the bleeding conduit 110 and the cuff 120.

A material (for example MPC polymer) having blood compatibility and antithrombogenicity may be applied to an outer peripheral surface of the bleeding conduit by coating by way of the above-mentioned first gas non-permeable material layer.

### 2. Method of Attaching Bleeding Conduit Assembly to Heart

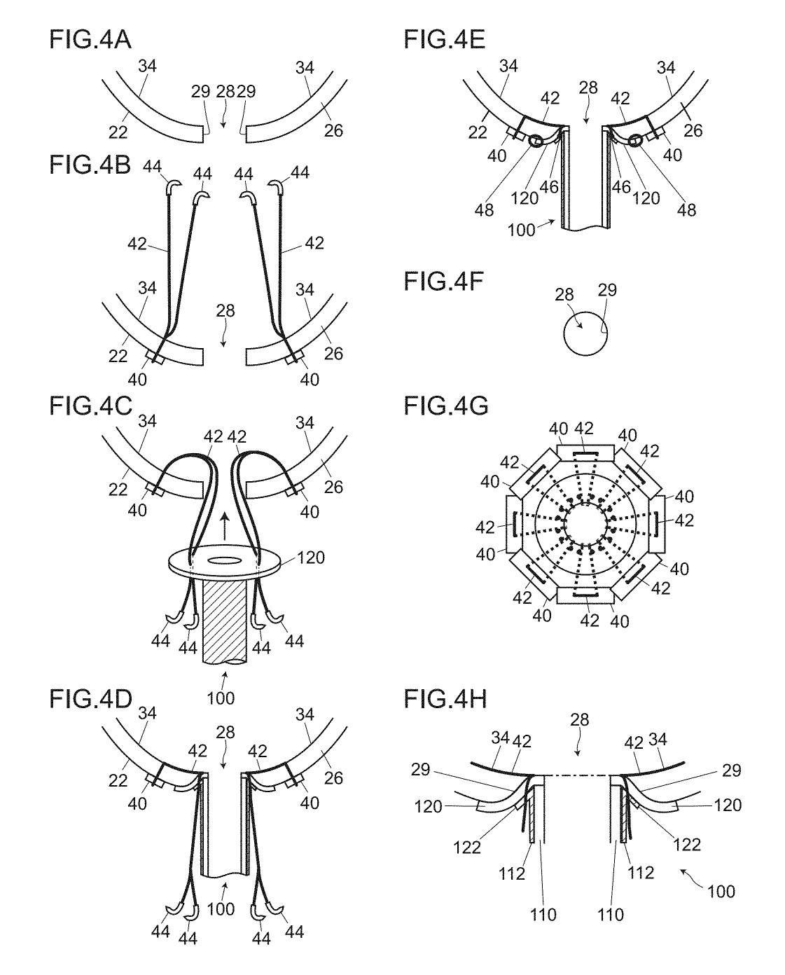

FIG. 4A to FIG. 4H are views for schematically describing a mode where the bleeding conduit assembly 100 according to the embodiment 1 is attached to an apex cordis of a heart left ventricle. FIG. 4A to FIG. 4E are views showing respective steps, wherein FIG. 4F is a plan view corresponding to FIG. 4A, FIG. 4G is a plan view corresponding to FIG. 4C, and FIG. 4H is a partially enlarged view corresponding to FIG. 4D.

Mounting of the bleeding conduit assembly 100 according to the embodiment 1 on the apex cordis of the heart left ventricle is performed in accordance with the following steps as shown in FIG. 4A to FIG. 4H.

### (1) First Step

Firstly, the bleeding conduit assembly 100 is prepared (see FIG. 2A to FIG. 2C, FIG. 3A and FIG. 3B). The bleeding conduit assembly 100 is formed of: the bleeding conduit 110 made of a porous material; the cuff 120 mounted on one end portion of the bleeding conduit 110; and the reinforcing ring (auxiliary PTFE helix) 130 welded to the outer peripheral portion of the bleeding conduit 110. The first gas non-permeable material layer 112 is formed on the outer peripheral surface of the bleeding conduit 110, and the second gas non-permeable material layer 122 is formed on an inner-peripheral-side predetermined region on a surface of the cuff 120 on a bleeding conduit side.

### (2) Second Step

Next, an opening (a punched hole in this case) 28 is formed in a predetermined portion of the heart (the apex cordis 24 of the left ventricle 22) by punching using a puncher (see FIG. 4A and FIG. 4F).

### (3) Third Step

Next, with respect to each of a plurality of respective pledgets 40 disposed at positions surrounding the opening 28 outside the heart, suture thread bridging is performed to the heart in such a manner that, using a suture thread 42 having needles 44 on both ends, the respective needles 44 are made to penetrate myocardium 26 from a surface of the pledget 40 and to reach the inside of the heart (see FIG. 4B).

### (4) Fourth Step

Next, with respect to the respective suture threads 42, the respective needles 44 are taken out to the outside of the heart through the opening 28, and the respective needles 44 are made to pass through the cuff 120 of the bleeding conduit assembly 100 thus performing suture thread bridging to the cuff 120 (see FIG. 4C and FIG. 4G).

### (5) Fifth Step

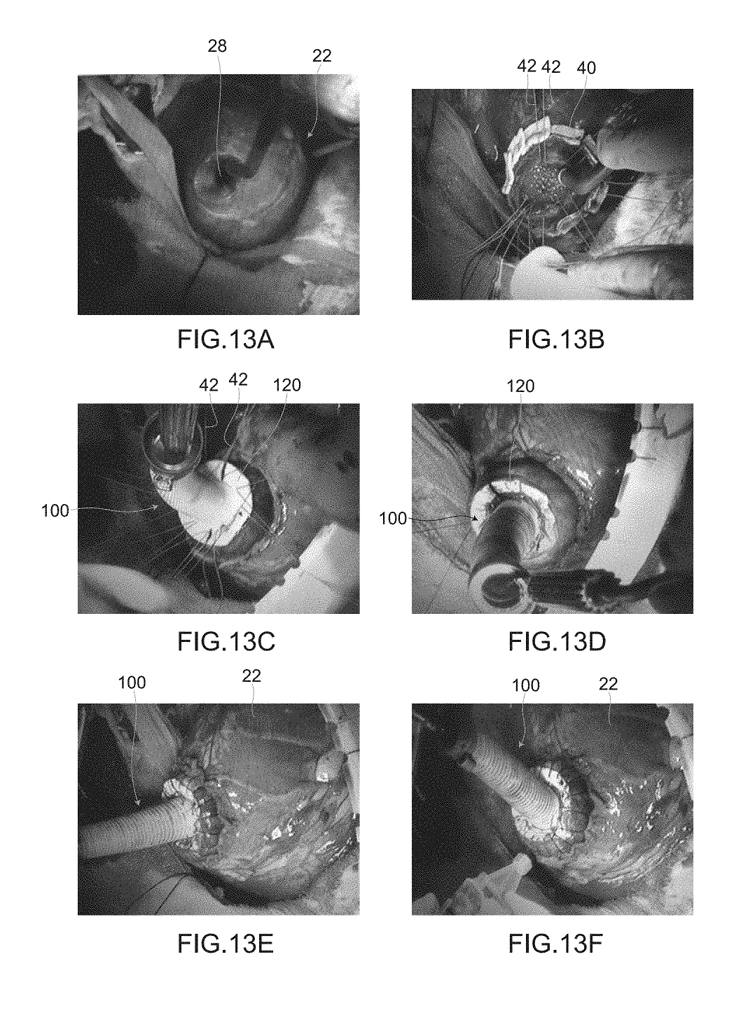

Next, in a state where a surface of the bleeding conduit 110 and an inner wall surface 34 of the heart become coplanar with each other, that is, in a state where the bleeding conduit does not project from the inner wall surface of the heart by tying the respective suture threads 42, the bleeding conduit assembly 100 is attached to the heart (see FIG. 4D, FIG. 4H and FIG. 13D). In the fifth step, for attaching the bleeding conduit assembly 100 to the heart in a state where the surface of the bleeding conduit 110 and the inner wall surface 34 of the heart become coplanar with each other, the respective suture threads are tied so as to bring about a state where the cuff covers a punching cut surface 29 of the opening 28 formed in the heart by an operation (see FIG. 4H).

### (6) Sixth Step

Finally, within a region which surrounds the opening 28, suture thread bridging is performed in a spiral shape between the cuff 120 (an outer peripheral portion of the cuff 120) and the heart left ventricle 22 using suture threads different from the above-mentioned suture threads thus attaching the bleeding conduit assembly 100 to the heart more firmly (see FIG. 4E). In this case, suture thread bridging may be performed between the cuff 120 and regions sandwiched between the cuff 120 and the pledgets 40. Alternatively, suture thread bridging may be performed between the cuff 120 and the pledgets 40 or between the cuff 120 and a region surrounding the pledgets 40.

In an operation performed for attaching the bleeding conduit assembly 100 according to the embodiment 1 to the heart, monofilament suture threads made of PTFE, for example are used. Multifilament suture threads made of PTFE, multifilament suture threads made of polyester or the like can be also used in place of monofilament suture threads made of PTFE.

By performing the above-mentioned steps sequentially, the bleeding conduit assembly 100 according to the embodiment 1 can be attached to the apex cordis 24 of the heart left ventricle 22.

### 3. Advantageous Effects of Embodiment 1

The bleeding conduit assembly 100 according to the embodiment 1 is formed of the bleeding conduit 110 and the cuff 120 mounted on one end portion of the bleeding conduit 110 and hence, an inflow cannula (cannula chip) does not exist. In this manner, the bleeding conduit assembly 100 according to the embodiment 1 does not include the inflow cannula. Accordingly, it is possible to prevent the occurrence of

a state where thrombus occurs at a root portion of the inflow cannula in the case where the bleeding conduit assembly is attached to a heart of a patient having an extremely weak blood flow in the heart,

a state where thrombus occurs at a portion where the inflow cannula and a myocardium are disposed close to each other or are brought into contact with each other in the case where the bleeding conduit assembly is brought into a state where the bleeding conduit assembly is attached to a heart obliquely with respect to the heart due to an operation or contraction of the heart after an operation, and

a state where even in the case where a pressure in a heart chamber becomes a negative pressure (less than 1 atmospheric pressure), the blood flow 30 becomes weak so that thrombus 32 occurs at a root portion of the inflow cannula 920 or thrombus 32 occurs at a portion where the inflow cannula 920 and the myocardium 26 are disposed close to each other or are brought into contact with each other. That is, it is possible to overcome the above-mentioned three drawbacks which the above-mentioned conventional bleeding conduit assembly has.

According to the bleeding conduit assembly 100 of the embodiment 1, the bleeding conduit 110 is made of a porous material. Accordingly, in a step of using the bleeding conduit (bleeding conduit assembly) , thrombus is anchored on an inner peripheral surface of the bleeding conduit made of a porous material, at least a pseudo inner membrane (quasi inner membrane) is formed in such a portion, and endothelial cells are stably fixed to a part near a heart (a part within 2 to 3 cm from the heart). As a result, it is possible to provide the bleeding conduit assembly which can reduce a problem of the occurrence of thrombus.

According to the bleeding conduit assembly 100 of the embodiment 1, the first gas non-permeable material layer 112 is formed on the outer peripheral surface of the bleeding conduit 110. Accordingly, in spite of the fact that the bleeding conduit made of a porous material is used, it is possible to prevent entrainment of air into the bleeding conduit even when a pressure in the bleeding conduit becomes a negative pressure (less than 1 atmospheric pressure).

According to the bleeding conduit assembly 100 of the embodiment 1, the bleeding conduit 110 is formed of an artificial blood vessel made of ePTFE having excellent flexibility and hence, the bleeding conduit assembly can be easily connected to the ventricular assist pump. According to the bleeding conduit assembly 100 of the embodiment 1, the bleeding conduit 110 is formed of an artificial blood vessel made of ePTFE having excellent flexibility and hence, a burden imposed on a human body can be reduced. According to the bleeding conduit assembly 100 of the embodiment 1, the bleeding conduit 110 is formed of an artificial blood vessel made of ePTFE and hence, it is possible to provide the bleeding conduit assembly having excellent blood compatibility and excellent antithrombogenicity.

According to the bleeding conduit assembly 100 of the embodiment 1, the cuff 120 is made of a PTFE non-woven fabric and hence, it is possible to provide the bleeding conduit assembly having excellent biocompatibility with a heart tissue. According to the bleeding conduit assembly 100 of the embodiment 1, the cuff 120 is made of a PTFE non-woven fabric and hence, the cuff has high flexibility whereby the degree of freedom of an operation performed at the time of attaching the bleeding conduit assembly to a heart is increased. It is possible to realize the bleeding conduit assembly having a shape which conforms to a shape and a structure of a heart of a patient. Further, in performing an operation, thread bridging is performed to pledgets, the heart, and the cuff respectively using suture threads and, thereafter, the suture threads are tied. Accordingly, it is possible to realize the bleeding conduit assembly having a shape such that the cuff covers a cross section (for example, a punching cut surface) of an opening formed in a heart by an operation (see FIG. 4D, FIG. 4E, and FIG. 4H). In the bleeding conduit assembly 100 of the embodiment 1, the cuff 120 is not limited to a cuff made of a PTFE non-woven fabric, and may be a cuff made of a polyester non-woven fabric or the like.

According to the bleeding conduit assembly 100 of the embodiment 1, the first gas non-permeable material layer 112 is formed of a polyurethane layer or a polycarbonate urethane layer and hence, the bleeding conduit assembly 100 can acquire an excellent gas non-permeable performance. As a result, the bleeding conduit assembly 100 can acquire an excellent air entrainment prevention performance. The first gas non-permeable material layer 112 is formed of a polyurethane layer or a polycarbonate urethane layer and hence, the first gas non-permeable material layer 112 does not adversely affect a living body.

According to the bleeding conduit assembly 100 of the embodiment 1, the second gas non-permeable material layer 122 is formed in at least the inner-peripheral-side predetermined region on the surface of the cuff 120 on a bleeding conduit side. Accordingly, as shown in FIG. 4E, when the bleeding conduit assembly 100 is attached to a heart, it is possible to suppress as much as possible the occurrence of a state where air is entrained through the cuff 120.

According to the bleeding conduit assembly 100 of the embodiment 1, a thickness of the polycarbonate urethane layer which forms the first gas non-permeable material layer 112 is set to 0.05 mm or more and hence, the bleeding conduit assembly 100 has a high gas non-permeable performance and hence, the bleeding conduit assembly 100 can acquire a sufficient air entrainment prevention effect. Further, the thickness of the polycarbonate urethane layer is set to 0.5 mm or less and hence, the bleeding conduit assembly 100 has high flexibility. Accordingly, in connecting the bleeding conduit assembly to the ventricular assist pump, operability is not lowered and, at the same time, a burden on a human body is not increased.

According to the bleeding conduit assembly 100 of the embodiment 1, the first gas non-permeable material layer 112 and the second gas non-permeable material layer 122 are formed continuously. Accordingly, when the bleeding conduit assembly 100 is attached to a heart, it is possible to suppress as much as possible the occurrence of a state where air is entrained from the inside of a heart through the cuff 120.

According to the bleeding conduit assembly 100 of the embodiment 1, the second gas non-permeable material layer 122 is formed within the region of at least 2 mm from the outer peripheral surface of the bleeding conduit 110. Accordingly, when the bleeding conduit assembly 100 is attached to a heart, it is possible to narrow as much as possible a path when air is entrained from the inside of a heart through the cuff 120.

According to the bleeding conduit assembly 100 of the embodiment 1, the second gas non-permeable material layer 122 is not formed within the region of at least 2 mm from the outer periphery of the cuff 120. Accordingly, when the bleeding conduit assembly 100 is attached to a heart, a tissue of a living body infiltrates into the cuff also on a surface of the cuff 120 on a bleeding conduit side and hence, the cuff and the living body are favorably adhered to each other.

According to the bleeding conduit assembly 100 of the embodiment 1, the second gas non-permeable material layer 122 is formed of a polyurethane layer or a polycarbonate urethane layer and hence, the bleeding conduit assembly can acquire an excellent gas non-permeable performance. As a result, the bleeding conduit assembly can acquire an excellent air entrainment prevention performance. The second gas non-permeable material layer 122 is formed of a polyurethane layer or a polycarbonate urethane layer and hence, the second gas non-permeable material layer 122 does not adversely affect a living body.

According to the bleeding conduit assembly 100 of the embodiment 1, the cuff 120 is sutured to the bleeding conduit 110 and hence, flexible connection between the cuff 120 and the bleeding conduit 110 can be realized. Accordingly, the bleeding conduit assembly having a shape which conforms to a shape and a structure of a heart of a patient can be realized.

According to the bleeding conduit assembly 100 of the embodiment 1, a thickness of the polycarbonate urethane layer which forms the second gas non-permeable material layer 122 is set to 0.05 mm or more and hence, the bleeding conduit assembly 100 has a high gas non-permeable performance and hence, the bleeding conduit assembly 100 can acquire a sufficient air entrainment prevention effect. Further, the thickness of the polycarbonate urethane layer is set to 0.5 mm or less and hence, the cuff has high flexibility. Accordingly, it is possible to prevent the occurrence of a situation where operability in attaching the bleeding conduit assembly to a heart is lowered and a situation where it is difficult to realize a state that the cuff covers a cross section (for example, a punching cut surface) of the opening formed in the heart by an operation.

According to the bleeding conduit assembly 100 of the embodiment 1, a width W of the cuff 120 along a diametric direction is set to 5 mm or more. Accordingly, the cuff can sufficiently cover a cross section (punching surface) of the opening 28 formed in the heart by an operation (see FIG. 4D and FIG. 4H). Further, a suture bridging operation in a spiral shape can be performed between the heart and the cuff 120 (see FIG. 4E). On the other hand, the width W of the cuff 120 in a diametric direction is set to 16 mm or less and hence, undesired burden is not imposed on a body of a patient.

In the bleeding conduit assembly 100 of the embodiment 1, the bleeding conduit assembly 100 is a bleeding conduit assembly which is attached to an apex cordis of a left ventricle of the heart or an area in the vicinity of the apex cordis, and is used for introducing blood in the left ventricle of the heart into the ventricular assist pump. Accordingly, the bleeding conduit assembly can be suitably used in a ventricular assist system which can efficiently assist a heart.

According to the bleeding conduit assembly 100 of the embodiment 1, the annular reinforcing member (reinforcing ring 130) is disposed at at least one end portion of the bleeding conduit 110 and hence, it is possible to prevent the occurrence of a situation where an opening formed in a heart by an operation is gradually narrowed after the operation.

According to the bleeding conduit assembly 100 of the embodiment 1, the reinforcing ring 130 disposed along a longitudinal direction of the artificial blood vessel is directly used as the annular reinforcing member. Accordingly, it is unnecessary to use a new part as the annular reinforcing member and hence, it is possible to provide the bleeding conduit assembly having the small number of parts.

In the bleeding conduit assembly 100 of the embodiment 1, the connecting ring 140 for connecting the bleeding conduit 110 to the ventricular assist pump is disposed on the other end portion of the bleeding conduit 110 and hence, it is possible to provide the bleeding conduit assembly having excellent connection operability with the ventricular assist pump.

According to the bleeding conduit assembly 100 of the embodiment 1, the above-mentioned width W is set to 5 mm or more. Accordingly, the cuff can sufficiently cover a cross section (punching surface) of the opening 28 formed in the heart by an operation (see FIG. 4D and FIG. 4H) . Further, a suture bridging operation in a spiral shape can be performed between the heart and the cuff 120 (see FIG. 4E). On the other hand, the above-mentioned width W is set to 16 mm or less and hence, undesired burden is not imposed on a body of a patient.

The bleeding conduit assembly 100 of the embodiment 1 is a bleeding conduit assembly which is attached to the apex cordis 24 of the heart left ventricle 22 or an area in the vicinity of the apex cordis 24, and is used for introducing blood in the heart into the ventricular assist pump 10. Accordingly, the bleeding conduit assembly 100 can be suitably used in a ventricular assist system which can efficiently assist a heart.

The ventricular assist pump system 1 of the embodiment 1 includes the bleeding conduit assembly 100 of the embodiment 1. Accordingly, it is possible to provide the ventricular assist pump system capable of solving the previously mentioned three drawbacks which occur attributed to the presence of an inflow cannula (cannula chip) in the conventional bleeding conduit assembly.

According to the ventricular assist pump system 1 of the embodiment 1, the bleeding conduit 110 is made of a porous material. Accordingly, in a step of using the bleeding conduit (bleeding conduit assembly) , thrombus is anchored on an inner peripheral surface of the bleeding conduit made of a porous material so that a pseudo inner membrane (quasi inner membrane) is formed, and endothelial cells are stably fixed to a part near a heart (a part within 2 to 3 cm from the heart). As a result, it is possible to provide the ventricular assist pump system which can reduce a problem of the occurrence of thrombus compared to the prior art.

According to the ventricular assist pump system 1 of the embodiment 1, the first gas non-permeable material layer 112 is formed on the outer peripheral surface of the bleeding conduit 110. Accordingly, in spite of the fact that the bleeding conduit made of a porous material is used, it is possible to prevent entrainment of air into the bleeding conduit even when a pressure in the bleeding conduit becomes a negative pressure (less than 1 atmospheric pressure).

According to a method of attaching a bleeding conduit assembly to a heart of the embodiment 1, at least the above-mentioned first to fifth steps are performed in this order. Accordingly, by tying the respective suture threads in the fifth step, the bleeding conduit assembly can be properly attached to a heart in a state where a surface of the bleeding conduit and an inner wall surface of the heart become coplanar with each other, that is, in a state where the bleeding conduit does not project from the inner wall surface of the heart. Accordingly, it is possible to overcome the previously mentioned three drawbacks generated attributed to the presence of an inflow cannula (cannula chip) in the conventional bleeding conduit assembly. In the fifth step, for properly attaching the bleeding conduit assembly to the heart in a state where the surface of the bleeding conduit and the inner wall surface of the heart become coplanar with each other, it is preferable that the respective suture threads be tied so as to bring about a state where the cuff covers a punching cut surface of the opening formed in the heart by an operation.

According to the method of attaching a bleeding conduit assembly to a heart of the embodiment 1, the bleeding conduit assembly of the embodiment 1, that is, the bleeding conduit assembly which includes the bleeding conduit made of a porous material can be properly attached to a heart. Accordingly, in a step of using the bleeding conduit (bleeding conduit assembly), thrombus is anchored on an inner peripheral surface of the bleeding conduit made of a porous material so that a pseudo inner membrane (quasi inner membrane) is formed, and endothelial cells are stably fixed to a part near a heart (a part within 2 to 3 cm from the heart). As a result, it is possible to provide the ventricular assist pump system which can reduce a problem of the occurrence of thrombus compared to the prior art. Further, the bleeding conduit assembly according to the embodiment 1, that is, the bleeding conduit assembly where the first gas non-permeable material layer is formed on the outer peripheral surface of the bleeding conduit can be properly attached to the heart. Accordingly, in spite of the fact that the bleeding conduit made of a porous material is used, it is possible to prevent entrainment of air into the bleeding conduit even when a pressure in the bleeding conduit becomes a negative pressure (less than 1 atmospheric pressure).

According to the method of attaching a bleeding conduit assembly to a heart of the embodiment 1, the method further includes the above-mentioned sixth step after the fifth step. Accordingly, the bleeding conduit assembly can be attached to the heart more firmly.

### Embodiments 2 to 5

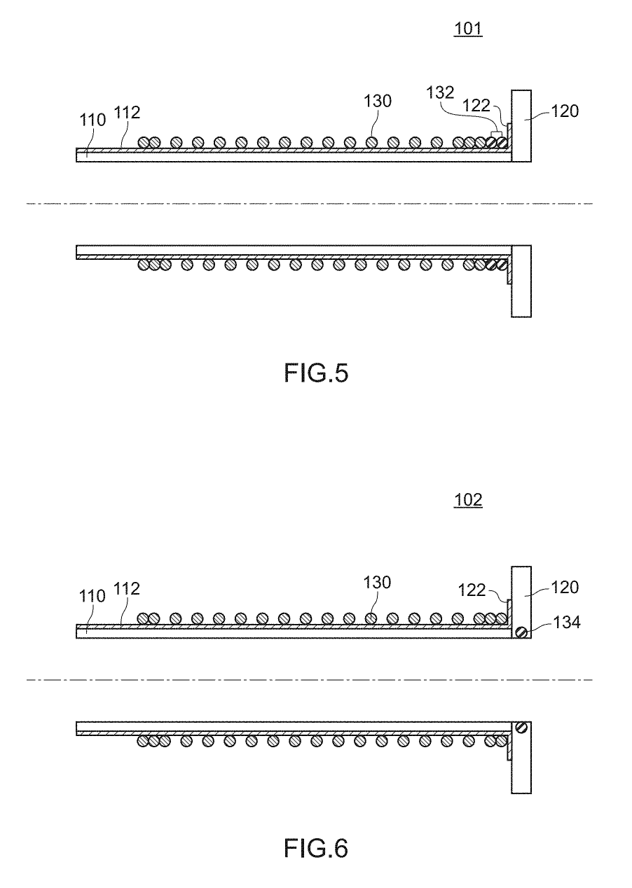

FIG. 5 is a view (cross-sectional view) for describing a bleeding conduit assembly 101 according to an embodiment 2.

FIG. 6 is a view (cross-sectional view) for describing a bleeding conduit assembly 102 according to an embodiment 3.

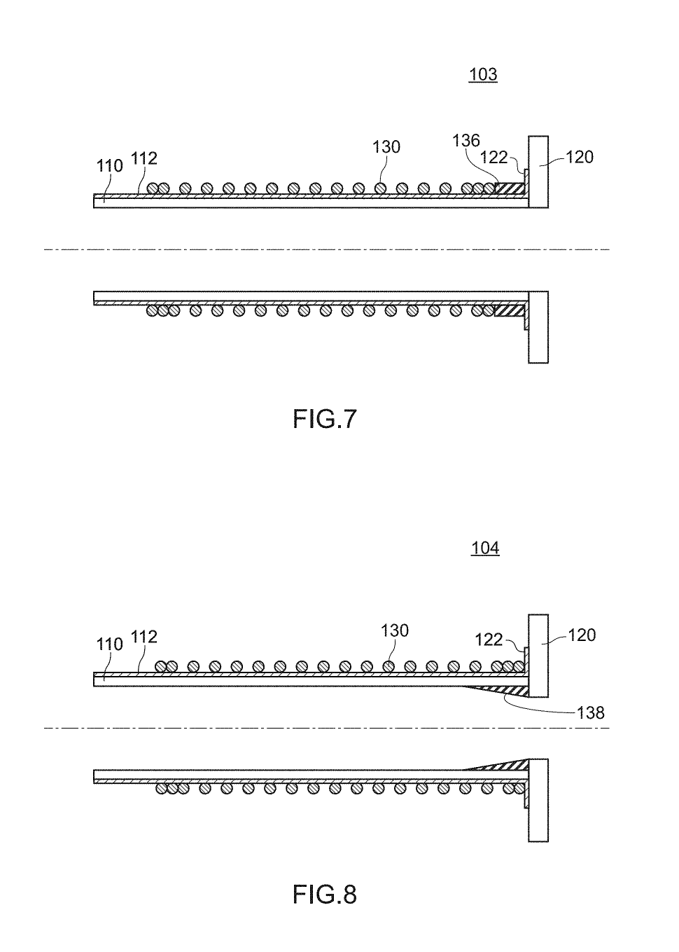

FIG. 7 is a view (cross-sectional view) for describing a bleeding conduit assembly 103 according to an embodiment 4.

FIG. 8 is a view (cross-sectional view) for describing a bleeding conduit assembly 104 according to an embodiment 5.

The bleeding conduit assemblies 101 to 104 according to the embodiments 2 to 5 basically have substantially the same configuration as the bleeding conduit assembly 100 according to the embodiment 1. However, the bleeding conduit assemblies 101 to 104 according to the embodiments 2 to 5 differ from the bleeding conduit assembly 100 according to the embodiment 1 with respect to a point that the bleeding conduit assemblies 101 to 104 according to the embodiments 2 to 5 respectively include an annular reinforcing member in addition to the reinforcing ring 130 as the annular reinforcing member.

That is, the bleeding conduit assembly 101 according to the embodiment 2 includes, as shown in FIG. 5, a reinforcing ring 132 as an annular reinforcing member in addition to a reinforcing ring 130. The bleeding conduit assembly 102 according to the embodiment 3 includes, as shown in FIG. 6, a reinforcing ring 134 embedded in an inner peripheral portion of a cuff 120 as an annular reinforcing member. The bleeding conduit assembly 103 according to the embodiment 4 includes, as shown in FIG. 7, a reinforcing belt 136 disposed on an outer peripheral side of a bleeding conduit 110 as an annular reinforcing member. The bleeding conduit assembly 104 according to the embodiment 5 includes, as shown in FIG. 8, a reinforcing belt 138 disposed on an inner peripheral side of a bleeding conduit 110 as an annular reinforcing member. The reinforcing belt 130 is formed into a tapered shape where an inner peripheral surface is gradually narrowed along a blood flow direction so as not to obstruct the flow of blood.

In this manner, the bleeding conduit assemblies 101, 102, 103, 104 according to the embodiment 2 to 5 differ from the bleeding conduit assembly 100 according to the embodiment 1 with respect to the point that the respective bleeding conduit assemblies 101, 102, 103, 104 include the annular reinforcing member in addition to the reinforcing ring 130 as the annular reinforcing member.

On the other hand, the bleeding conduit assemblies according to the embodiments 2 to 5 do not include the inflow cannula (cannula chip). Accordingly, in the same manner as the bleeding conduit assembly 100 according to the embodiment 1, it is possible to prevent the occurrence of

a state where thrombus occurs at a root portion of the inflow cannula in the case where the bleeding conduit assembly is attached to a heart of a patient having an extremely weak blood flow in the heart,

a state where thrombus occurs at a portion where the inflow cannula and a myocardium are disposed close to each other or are brought into contact with each other in the case where the bleeding conduit assembly is brought into a state where the bleeding conduit assembly is attached to a heart obliquely with respect to the heart due to an operation or contraction of the heart after an operation, and

a state where even in the case where a pressure in a heart chamber becomes a negative pressure (less than 1 atmospheric pressure), the blood flow 30 becomes weak so that thrombus 32 occurs at a root portion of the inflow cannula 920 or thrombus 32 occurs at a portion where the inflow cannula 920 and the myocardium 26 are disposed close to each other or are brought into contact with each other. That is, it is possible to overcome the above-mentioned three drawbacks.

In the bleeding conduit assemblies 101, 102, 103, 104 according to the embodiments 2 to 5, the bleeding conduit 110 is formed of a porous material. Accordingly, in the same manner as the bleeding conduit assembly 100 according to the embodiment 1, in a step of using the bleeding conduit (bleeding conduit assembly), thrombus is anchored on an inner peripheral surface of the bleeding conduit made of a porous material so that a pseudo inner membrane (quasi inner membrane) is formed, and endothelial cells are stably fixed to a part near a heart (a part within 2 to 3 cm from the heart). As a result, it is possible to reduce a problem of the occurrence of thrombus compared to the prior art.

According to the bleeding conduit assemblies 101, 102, 103, 104 of the embodiments 2 to 5, the first gas non-permeable material layer 112 is formed on the outer peripheral surface of the bleeding conduit 110. Accordingly, in the same manner as the bleeding conduit assembly 100 of the embodiment 1, in spite of the fact that the bleeding conduit made of a porous material is used, it is possible to prevent entrainment of air into the bleeding conduit even when a pressure in the bleeding conduit becomes a negative pressure (less than 1 atmospheric pressure).

In the bleeding conduit assemblies 101, 102, 103, 104 according to the embodiments 2 to 5, the reinforcing rings 132, 134 and the reinforcing belts 136, 138 may preferably be made of metal or a hard resin.

### Embodiment 6 to 9

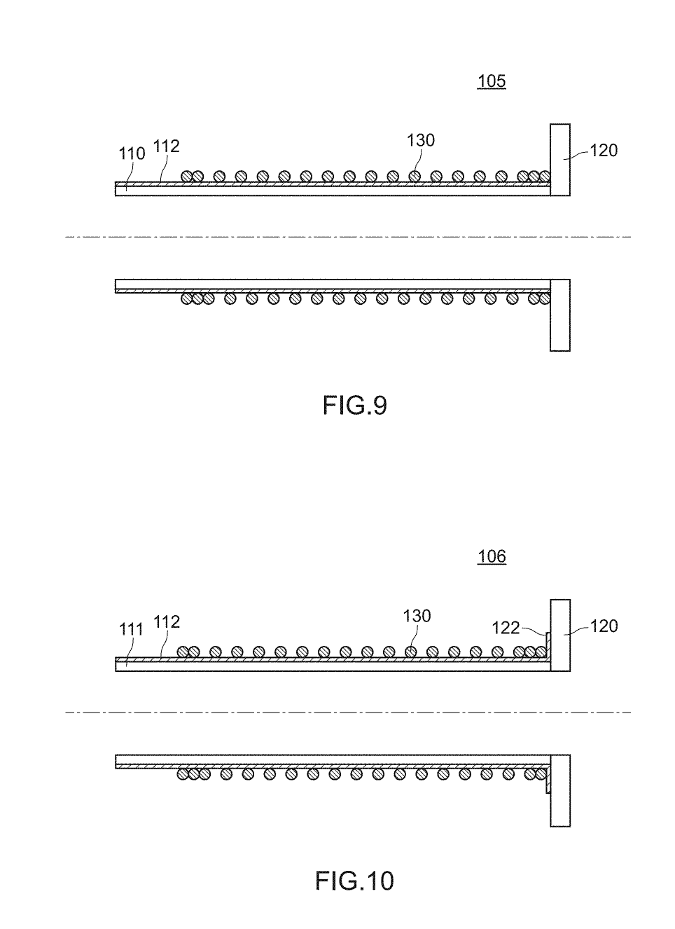

FIG. 9 is a view (cross-sectional view) for describing a bleeding conduit assembly 105 according to an embodiment 6.

FIG. 10 is a view (cross-sectional view) for describing a bleeding conduit assembly 106 according to an embodiment 7.

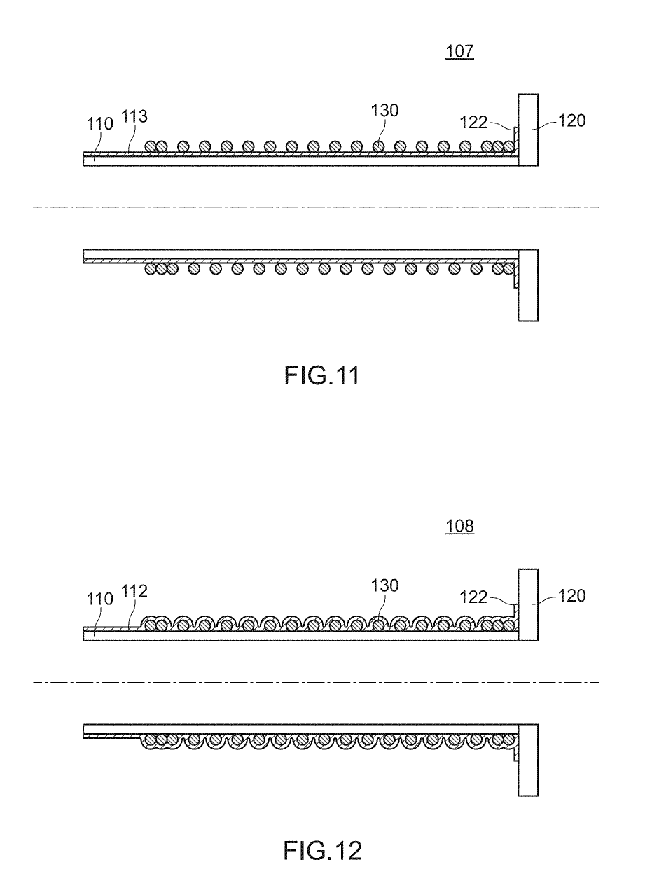

FIG. 11 is a view (cross-sectional view) for describing a bleeding conduit assembly 107 according to an embodiment 8.

FIG. 12 is a view (cross-sectional view) for describing a bleeding conduit assembly 108 according to an embodiment 9.

The bleeding conduit assembly 105 according to the embodiment 6 basically has substantially the same configuration as the bleeding conduit assembly 100 according to the embodiment 1. However, as shown in FIG. 9, the bleeding conduit assembly 105 according to the embodiment 6 differs from the bleeding conduit assembly according to the embodiment 1 with respect to a point that the bleeding conduit assembly 105 according to the embodiment 6 does not have the second gas non-permeable material layer. The bleeding conduit assembly 106 according to the embodiment 7 basically has substantially the same configuration as the bleeding conduit assembly 100 according to the embodiment 1. However, as shown in FIG. 10, the bleeding conduit assembly 106 according to the embodiment 7 differs from the bleeding conduit assembly 100 according to the embodiment 1 with respect to a point that a bleeding conduit 111 is formed of an artificial blood vessel made of a polyester woven fabric. The bleeding conduit assembly 106 according to the embodiment 8 basically has substantially the same configuration as the bleeding conduit assembly 100 according to the embodiment 1. However, as shown in FIG. 11, the bleeding conduit assembly 107 according to the embodiment 8 differs from the bleeding conduit assembly 100 according to the embodiment 1 with respect to a point that a first gas non-permeable material layer 113 is formed of a polyurethane layer. The bleeding conduit assembly 108 according to the embodiment 9 basically has substantially the same configuration as the bleeding conduit assembly 100 according to the embodiment 1. However, as shown in FIG. 12, the bleeding conduit assembly 108 according to the embodiment 9 differs from the bleeding conduit assembly 100 according to the embodiment 1 with respect to a point that a first gas non-permeable material layer 112 and a second gas non-permeable material layer 122 are formed in such a manner that an auxiliary helix 130 is disposed on (welded to) an outer peripheral surface of the bleeding conduit 110 and, thereafter, the first gas non-permeable material layer 112 and the second gas non-permeable material layer 122 are formed (applied by coating) on the outer peripheral surface of the bleeding conduit 110.

In this manner, the bleeding conduit assemblies 105, 106, 107, 108 according to the embodiments 6 to 9 respectively differ from the bleeding conduit assembly 100 according to the embodiment 1 with respect to the following points.

The point that the bleeding conduit assemblies 105, 106, 107, 108 do not have the second gas non-permeable material layer.

The point that the bleeding conduit 111 is formed of an artificial blood vessel made of a polyester woven fabric.

The point that the first gas non-permeable material layer 113 is formed of a polyurethane layer.

The point that the first gas non-permeable material layer 112 and the second gas non-permeable material layer 122 are formed in such a manner that the auxiliary helix 130 is disposed on the outer peripheral surface of the bleeding conduit 110 and, thereafter, the first gas non-permeable material layer 112 and the second gas non-permeable material layer 122 are formed on the outer peripheral surface of the bleeding conduit 110.

However, an inflow cannula (cannula chip) does not exist in the bleeding conduit assemblies 105, 106, 107, 108 according to the embodiments 6 to 9. Accordingly, in the same manner as the bleeding conduit assembly 100 according to the embodiment 1, it is possible to prevent the occurrence of

a state where thrombus occurs at a root portion of the inflow cannula in the case where the bleeding conduit assembly is attached to a heart of a patient having an extremely weak blood flow in the heart,

a state where thrombus occurs at a portion where the inflow cannula and a myocardium are disposed close to each other or are brought into contact with each other in the case where the bleeding conduit assembly is brought into a state where the bleeding conduit assembly is attached to a heart obliquely with respect to the heart due to an operation or contraction of the heart after an operation, and

a state where even in the case where a pressure in a heart chamber becomes a negative pressure (less than 1 atmospheric pressure), the blood flow 30 becomes weak so that thrombus 32 occurs at a root portion of the inflow cannula 920 or thrombus 32 occurs at a portion where the inflow cannula 920 and the myocardium 26 are disposed close to each other or are brought into contact with each other. That is, it is possible to overcome the above-mentioned three drawbacks.

Further, according to the bleeding conduit assemblies 105, 106, 107, 108 of the embodiments 6 to 9, the bleeding conduit 110 is formed of a porous material. Accordingly, in the same manner as the bleeding conduit assembly 100 according to the embodiment 1, in a step of using the bleeding conduit (bleeding conduit assembly) , thrombus is anchored on an inner peripheral surface of the bleeding conduit made of a porous material so that a pseudo inner membrane (quasi inner membrane) is formed, and endothelial cells are stably fixed to a part near a heart (a part within 2 to 3 cm from the heart). As a result, it is possible to reduce a problem of the occurrence of thrombus compared to the prior art.

According to the bleeding conduit assemblies 105, 106, 107, 108 of the embodiments 6 to 9, the first gas non-permeable material layer 112 is formed on the outer peripheral surface of the bleeding conduit 110. Accordingly, in the same manner as the bleeding conduit assembly 100 of the embodiment 1, in spite of the fact that the bleeding conduit made of a porous material is used, it is possible to prevent entrainment of air into the bleeding conduit even when a pressure in the bleeding conduit becomes a negative pressure (less than 1 atmospheric pressure).

### [Test Example]

The test example is a test example for confirming that a bleeding conduit assembly according to the present invention can be used as a bleeding conduit assembly which is attached to a heart and used for introducing blood in the heart into a ventricular assist pump from the heart in the same manner as the conventional bleeding conduit assembly 900.

### 1. Mounting of Bleeding Conduit Assembly on Heart

FIG. 13A to FIG. 13F are photographs showing a mode where the bleeding conduit assembly 100 was attached to a heart in the test example. FIG. 13A to FIG. 13F are views showing respective operational steps.

Mounting of the bleeding conduit assembly on the heart is basically performed by a method substantially equal to the “method of attaching a bleeding conduit assembly to a heart” according to the embodiment 1.

That is, firstly, in a first step, the bleeding conduit assembly 100 was prepared (see FIG. 2A to FIG. 2C, FIG. 3A and FIG. 3B). The bleeding conduit assembly 100 was formed of: the bleeding conduit 110 made of a porous material; the cuff 120 mounted on one end portion of the bleeding conduit 110; the reinforcing ring (auxiliary PTFE helix) 130 welded to the outer peripheral portion of the bleeding conduit 110; and the connecting ring 140 disposed at the other end portion of the bleeding conduit 110. The first gas non-permeable material layer 112 was formed on the outer peripheral surface of the bleeding conduit 110, and the second gas non-permeable material layer 122 was formed at least on an inner-peripheral-side predetermined region on a surface of the cuff 120 on a bleeding conduit side. A urethane resin was not applied by coating to the inner peripheral surface of the bleeding conduit 110. Further, MPC coating is applied to neither the outer peripheral surface nor the inner peripheral surface of the bleeding conduit 110.

Next, in a second step, a chest of a calf (age: three months; weight: 83 kg; sex: male) was opened and, thereafter, an opening (a punched hole) 28 was formed in a predetermined portion of the heart (an apex cordis of a left ventricle) by punching using a puncher having a diameter of 19 mm (see FIG. 4A and FIG. 13A).

Next, in a third step, with respect to each of the plurality of respective pledgets 40 (10 pledgets 40) disposed at positions surrounding the opening 28 outside the heart, suture thread bridging was performed to the heart in such a manner that, using the suture thread 42 having needles 44 on both ends, the respective needles 44 were made to penetrate the myocardium 26 from a surface of the pledget 40 and to reach the inside of the heart (see FIG. 4B).

Next, in a fourth step, with respect to the respective suture threads 42, the respective needles 44 were taken out to the outside of the heart through the opening 28 (see FIG. 13B), and the respective needles 44 were made to pass through the cuff 120 of the bleeding conduit assembly 100 thus performing suture thread bridging to the cuff 120 (see FIG. 4C and FIG. 13C).

Next, in a fifth step, in a state where a surface of the bleeding conduit and an inner wall surface of the heart became coplanar with each other, that is, in a state where the bleeding conduit did not project from the inner wall surface of the heart by tying the respective suture threads 42, the bleeding conduit assembly 100 was attached to the heart (see FIG. 4D, FIG. 4E and FIG. 13D). In the fifth step, for attaching the bleeding conduit assembly 100 to the heart in a state where the surface of the bleeding conduit 110 and the inner wall surface 34 of the heart became coplanar with each other, the respective suture threads are tied so as to bring about a state where the cuff covers a punching cut surface 29 of the opening 28 formed in the heart by an operation (see FIG. 4H).

Next, in a sixth step, within a region sandwiched by the pledget 40 and the opening 28, suture thread bridging was performed in a spiral shape between the heart and the cuff 120 using suture threads 48 different from the suture threads 42 thus attaching the bleeding conduit assembly to the heart more firmly (see FIG. 4E and FIG. 13E).

Next, a fibrin adhesive was applied by coating to the whole bleeding conduit assembly 100 (see FIG. 13F). Next, the bleeding conduit assembly 100 was connected to a blood introducing portion of the ventricular assist pump 10, and the outflow graft 12 which was attached to an arterial in advance was connected to a blood discharging portion of the ventricular assist pump 10. After the ventricular assist pump 10 was operated, a chest of a patient was closed. A ventricular assist pump (EVAHEART CO2 system) of SUN MEDICAL TECHNOLOGY RESEARCH was used as the ventricular assist pump 10.

### 2. Evaluation Method

### (1) Evaluation Method 1

The evaluation method 1 is an evaluation method whether or not attaching a bleeding conduit assembly to a heart can be performed properly using the bleeding conduit assembly according to the test example. The evaluation in accordance with the evaluation method 1 was conducted based on determination of a doctor who has conducted an operation.

### (2) Evaluation Method 2

The evaluation method 2 is an evaluation method whether or not a bleeding conduit assembly according to the test example can be used as a bleeding conduit assembly which is attached to a heart and used for introducing blood in the heart into a ventricular assist pump from the heart in the same manner as a conventional bleeding conduit assembly. The evaluation in accordance with the evaluation method 2 was performed: by observing a state of a calf after an operation of embedding a ventricular assist pump in a body of the calf; by observing a state of the bleeding conduit assembly after bringing the calf into a sacrificial death state after a lapse of 60 days from the operation of embedding the ventricular assist pump and; and by observing renal infarction in cross section of a liver.

### 3. Evaluation Result

### (1) Evaluation Result 1

As a result of the evaluation performed in accordance with the evaluation method 1, it was confirmed that attaching the bleeding conduit assembly to the heart can be properly performed using the bleeding conduit assembly according to the test example (see FIG. 13A to FIG. 13F).

### (2) Evaluation Result2

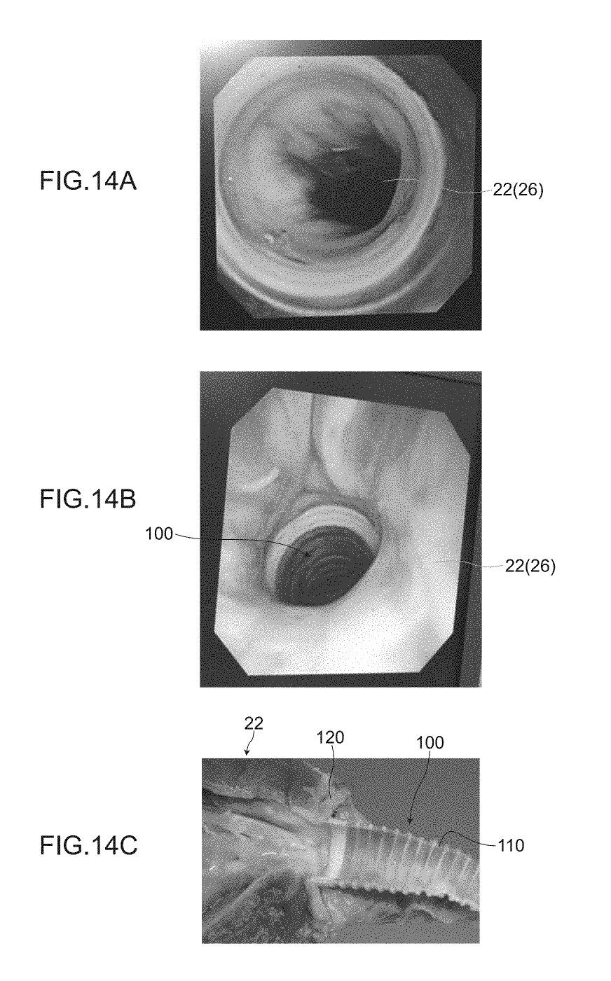

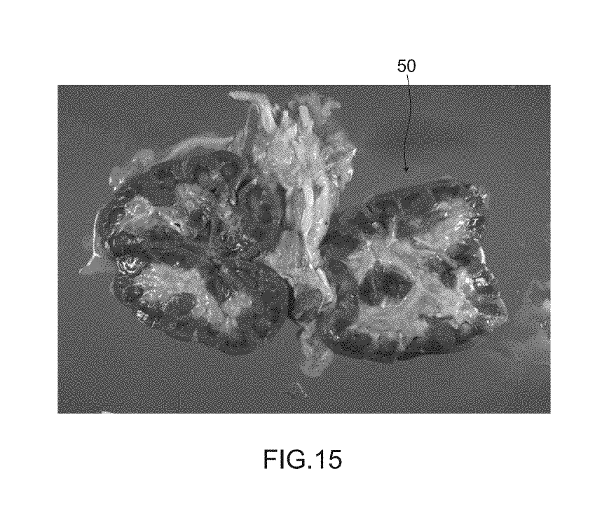

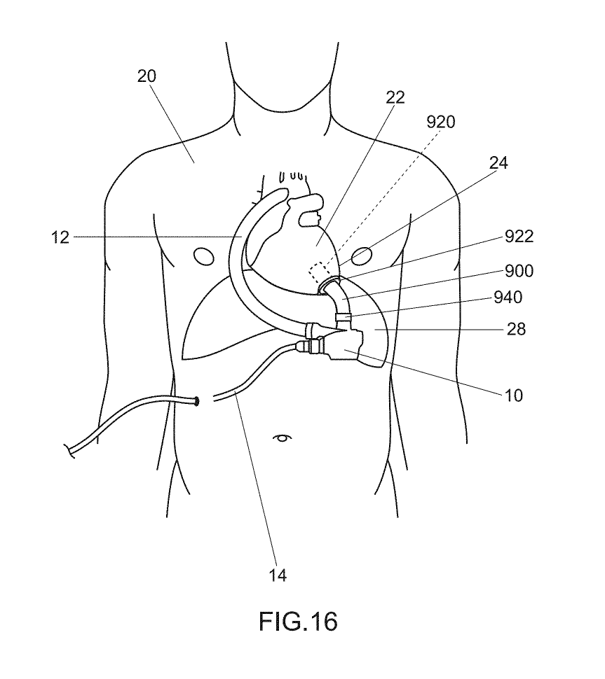

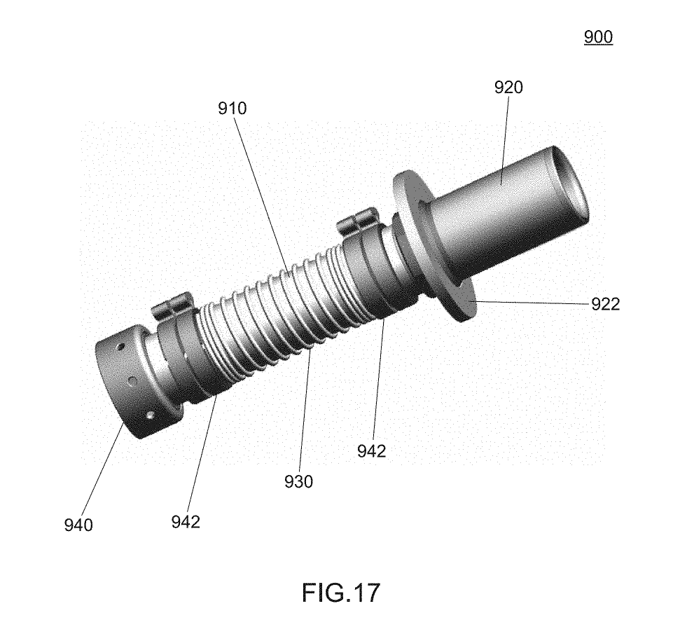

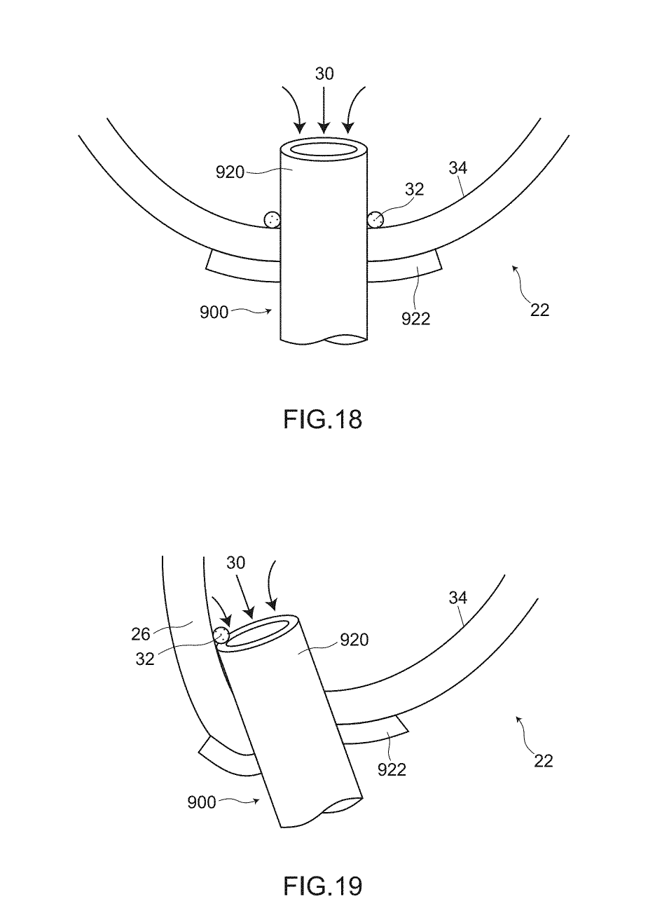

FIG. 14A to FIG. 14C are photographs of the bleeding conduit assembly 100 in the test example after a calf was brought into a sacrificial death state after a lapse of 60 days from attaching the bleeding conduit assembly 100 to a heart of the calf. FIG. 14A is a photograph of the inside of the bleeding conduit assembly taken using an endoscope from an artificial blood vessel side. FIG. 14B is a photograph of the inside of the bleeding conduit assembly taken using an endoscope from a heart side. FIG. 14C is a photograph of a longitudinal cross section of the bleeding conduit assembly as viewed from a cross section side. FIG. 15 is a photograph showing a cross section of a liver of a calf in the test example after the calf was brought into a sacrificial death state after a lapse of 60 days from attaching the bleeding conduit assembly 100 to a heart of the calf.

A state of the calf after the operation of embedding the ventricular assist pump was favorable until the calf was brought into a sacrificial death (until 60 days elapsed after attaching the bleeding conduit assembly 100 to the heart). Further, as can be also understood from FIG. 14A to FIG. 14C and FIG. 15, even after 60 days elapsed after the operation of embedding the ventricular assist pump, the followings were confirmed.

The bleeding conduit assembly 100 was properly attached to the heart.

A smooth junction was formed between the bleeding conduit assembly 100 and the heart.

The remarkable increase of neoplasia which has a risk of being scattered was not recognized also at a connecting portion of the bleeding conduit assembly 100.

Closing of the opening (punched hole) 28 by cell proliferation was not recognized.

The occurrence of thrombus at the bleeding conduit assembly 100 or in the vicinity of the bleeding conduit assembly 100 was not recognized.

Renal infarction attributed to embedding of the ventricular assist pump 10 was not recognized.

Accordingly, it was confirmed that the bleeding conduit assembly according to the test example can be properly used as a bleeding conduit assembly which is attached to a heart and used for introducing blood in the heart into a ventricular assist pump from the heart in the same manner as the conventional bleeding conduit assembly. Since an MPC was not applied by coating to an inner peripheral surface of the bleeding conduit (artificial blood vessel) 110, a pseudo inner-film-like tissue was recognized (see FIG. 14C).

The present invention is not limited to the above-mentioned embodiments, and can be carried in various modifications without departing from the gist of the present invention. For example, the following modifications can be also carried out.

(1) In the above-mentioned respective embodiments, the present invention has been described by taking the case where the bleeding conduit assembly was attached to the apex cordis of the heart left ventricle as an example. However, the present invention is not limited to such a case. For example, the present invention is also applicable to the case where the bleeding conduit assembly is attached to a portion in the vicinity of the apex cordis of the heart left ventricle or the case where the bleeding conduit assembly is attached to a portion other than the heart left ventricle.

(2) In the above-mentioned embodiment 1, the opening 28 (that is, the punched hole) is formed in the predetermined portion of the heart (the apex cordis 24 of the left ventricle 22) by punching using a puncher. However, the present invention is not limited to such a case. The opening may be formed in the predetermined portion of the heart by a method other than punching such as a method which uses a coring knife, a surgical knife or scissors.

(3) In the above-mentioned respective embodiments, the connecting ring is used as a means for connecting the bleeding conduit assembly to the ventricular assist pump. However, the present invention is not limited to such a configuration. For example, threads may be formed on the other end portion of the bleeding conduit assembly, and the threads may be used as a means for connecting the bleeding conduit assembly to the ventricular assist pump.

(4) In the above-mentioned respective embodiments, the present invention has been described by taking the chipless bleeding conduit assembly where an inflow cannula does not exist and the first gas non-permeable material layer is formed on the outer peripheral surface of the bleeding conduit as an example. However, the present invention is not limited to such a configuration. For example, even with respect to a bleeding conduit assembly having the conventional structure where an inflow cannula exists and a first gas non-permeable material layer is formed on an outer peripheral surface of the bleeding conduit, the first gas non-permeable material layer is formed on the outer peripheral surface of the bleeding conduit and hence, even in the case where the bleeding conduit made of a porous material is used, it is possible to acquire an advantageous effect that entrainment of air can be prevented even when a pressure in the bleeding conduit becomes a negative pressure (less than 1 atmospheric pressure).

1. A bleeding conduit assembly attached to a heart and used for introducing blood in the heart into a ventricular assist pump from the heart, the bleeding conduit assembly comprising:
a bleeding conduit made of a porous material; and
a cuff mounted on one end portion of the bleeding conduit, wherein
a first gas non-permeable material layer is formed on an outer peripheral surface of the bleeding conduit.

2. The bleeding conduit assembly according to claim 1, wherein the bleeding conduit is formed of an artificial blood vessel made of a stretchable porous polytetrafluoroethylene.

3. The bleeding conduit assembly according to claim 1, wherein the bleeding conduit is formed of an artificial blood vessel made of a polyester woven fabric.

4. The bleeding conduit assembly according to claim 1, wherein the cuff is made of a polytetrafluoroethylene non-woven fabric.

5. The bleeding conduit assembly according to claim 1, wherein the first gas non-permeable material layer is formed of a polyurethane layer or a polycarbonate urethane layer.

6. The bleeding conduit assembly according to claim 1, wherein a second gas non-permeable material layer is formed in at least an inner-peripheral-side predetermined region on a surface of the cuff on a bleeding conduit side.

7. The bleeding conduit assembly according to claim 6, wherein the first gas non-permeable material layer and the second gas non-permeable material layer are formed continuously.

8. The bleeding conduit assembly according to claim 6, wherein the second gas non-permeable material layer is formed within a region of at least 2 mm from an outer peripheral surface of the bleeding conduit.

9. The bleeding conduit assembly according to claim 8, wherein the second gas non-permeable material layer is not formed within a region of at least 2 mm from an outer periphery of the cuff.

10. The bleeding conduit assembly according to claim 6, wherein the second gas non-permeable material layer is formed of a polyurethane layer or a polycarbonate urethane layer.

11. The bleeding conduit assembly according to claim 1, wherein the cuff is sutured to the bleeding conduit.

12. The bleeding conduit assembly according to claim 1, wherein an annular reinforcing member is disposed on at least one end portion of the bleeding conduit.

13. The bleeding conduit assembly according to claim 1, wherein a connecting ring for connecting the bleeding conduit to the ventricular assist pump is disposed on the other end portion of the bleeding conduit.

14. A bleeding conduit assembly attached to a heart and used for introducing blood in the heart into a ventricular assist pump from the heart, the bleeding conduit assembly comprising:
a bleeding conduit made of a porous material;
an inflow cannula disposed on one end portion of the bleeding conduit; and
a cuff disposed on a predetermined position of an outer periphery of the inflow cannula, wherein
a gas non-permeable material layer is formed on an outer peripheral surface of the bleeding conduit.

15. A ventricular assist pump system comprising:
a ventricular assist pump having a blood introducing portion and a blood discharging portion;
a bleeding conduit assembly according to claim 1, the bleeding conduit assembly connecting the blood introducing portion of the ventricular assist pump and a heart to each other; and
an outflow graft connecting the blood discharging portion of the ventricular assist pump and an aorta to each other.

16. A method of attaching a bleeding conduit assembly to a heart comprising in the following order:
a first step where a bleeding conduit assembly which is formed of a bleeding conduit formed of an artificial blood vessel and a cuff mounted on one end portion of the bleeding conduit is prepared;
a second step where an opening is formed at a predetermined portion of a heart;
a third step where a plurality of pledgets are disposed at a position where the pledgets surround the opening outside the heart, suture threads each having a needle on both ends respectively are prepared, the respective needles of the suture threads are made to penetrate a myocardium of the heart from surfaces of the pledgets and to reach the inside of the heart thus bridging the suture threads to the heart;
a fourth step where the respective needles of each suture thread are made to pass through the opening and are taken out to the outside of the heart, and the respective needles are made to pass through the cuff of the bleeding conduit assembly so as to perform bridging of the suture threads to the cuff with respect to the respective suture threads; and
a fifth step where the bleeding conduit assembly is attached to the heart in a state where a surface (plane) of the bleeding conduit and an inner wall surface of the heart become coplanar with each other by tying the respective suture threads.

17. The method of attaching a bleeding conduit assembly to a heart according to claim 16, wherein the bleeding conduit assembly is attached to the heart in a state where the bleeding conduit does not protrude to the inside of the heart from an inner wall surface of the heart in the fifth step.

18. The method of attaching a bleeding conduit assembly to a heart according to claim 16, wherein in the fifth step, the bleeding conduit assembly is attached to the heart in a state where the cuff covers a punching cut surface of the opening.

19. The method of attaching a bleeding conduit assembly to a heart according to claim 16, further comprising, after the fifth step, a sixth step where bridging of the suture threads is performed in a spiral shape between the cuff and the heart using suture threads different from the above-mentioned suture threads within a region surrounding the opening thus enabling more firm attaching the bleeding conduit assembly to the heart.
